(19) United States
(12) Patent Application Publication
(10) Pub. No.: US 2026/0090709 A1
(43) Pub. Date: Apr. 2, 2026

(54) EVALUATING SPECTROPOLARIMETRIC DATA PACKAGES OF AN EYE FOR MARKERS OF DISEASE

(71) Applicant: RETISPEC INC., Toronto (CA)

(72) Inventors: Eliav Shaked, Toronto (CA); Alon Hazan, Toronto (CA); Tommaso Alterini, Toronto (CA); Joni Petteri Teikari, Toronto (CA)

(73) Assignee: RETISPEC INC., Toronto (CA)

(21) Appl. No.: 19/214,892

(22) Filed: May 21, 2025

Related U.S. Application Data
(63) Continuation of application No. PCT/IB2023/000705, filed on Nov. 22, 2023.
(60) Provisional application No. 63/427,708, filed on Nov. 23, 2022.

(51) Int. Cl. A61B 3/00; A61B 3/12; A61B 5/00; G06T 7/00; G06T 7/11; G06V 10/26; G06V 10/764; G06V 10/766; G06V 10/82; G16H 50/20; G16H 50/30; G16H 50/70

(52) U.S. Cl. CPC A61B 3/0025; A61B 3/1225; A61B 5/004; A61B 5/0075; A61B 5/4088; A61B 5/4842; A61B 5/4848; A61B 5/7264; G06T 7/0012; G06T 7/11; G16H 50/20; G16H 50/30; G16H 50/70; A61B 2576/02; G06T 2207/10101; G06T 2207/20081; G06T 2207/20084; G06T 2207/30041; G06T 2207/30168; G06T 2207/30204; G06V 10/267; G06V 10/764; G06V 10/766; G06V 10/82; G06V 2201/03

(57) ABSTRACT

The disclosure relates to systems and methods for evaluating markers of disease by using optical techniques A method includes analyzing data from an imaging of an eye of a patient with at least one processor, the data from the imaging including a plurality of pixels and including, for each pixel, spatial, spectral, and polarimetric data generated from the imaging of the eye. The method further includes based on the analyzing, classifying the patient into at least one category of a plurality of categories, each category of the plurality of categories indicating a status with respect to a neurodegenerative disease that affects a central nervous system. The method further includes generating an output of one or more of the at least one category indicating the status of the patient with respect to the neurodegenerative disease that affects a central nervous system.

### CROSS-REFERENCE TO RELATED APPLICATION

This application is a bypass continuation application of PCT International Application No. PCT/IB2023/000705, filed Nov. 22, 2023, which claims the benefit of and priority to U.S. Provisional Patent Application No. 63/427,708, filed Nov. 23, 2022, the contents of each of which are incorporated herein by reference in their entirety.

### FIELD

This disclosure relates to systems and methods for evaluating markers of disease, for example, Alzheimer's disease, by using optical techniques.

### BACKGROUND

Early detection of neurological diseases, such as Alzheimer's disease (AD), for preventative treatment is difficult. Identifying neurological diseases involves either highly invasive procedures or imaging devices that are often inaccessible or inappropriate due to cost, complexity, or the use of harmful radioactive tracers.

### SUMMARY

The present disclosure relates to a method including: analyzing data from an imaging of an eye of a patient with at least one processor, the data from the imaging including a plurality of pixels and including, for each pixel, spatial, spectral, and polarimetric data generated from the imaging of the eye of the patient and wherein analyzing the data includes analyzing the spatial, spectral, and polarimetric data for the plurality of pixels: based on the analyzing, with the at least one processor, classifying the patient into at least one category of a plurality of categories, each category of the plurality of categories indicating a status with respect to a neurodegenerative disease that affects a central nervous system; and generating, with the at least one processor, an output of one or more of the at least one category indicating the status of the patient with respect to the neurodegenerative disease that affects a central nervous system.

In some embodiments, the present disclosure relates to a method, further including generating the spatial, spectral, and polarimetric data synchronously as a single data package of spectropolarimetric data. In some embodiments, the present disclosure relates to a method, wherein: the data from the imaging includes a multi-dimensional spectropolarimetric data package; and classifying the patient into the at least one category includes: applying, by the at least one processor, one or more neural networks to each dimension of the multi-dimensional spectropolarimetric data package to generate a dimensional output for each dimension of the multi-dimensional spectropolarimetric data package; and generating, by the at least one processor, the output as a disease classification of the multi-dimensional spectropolarimetric data package by combining each dimensional output of each dimension of the multi-dimensional spectropolarimetric data package. In some embodiments, the present disclosure relates to a method, wherein analyzing the data from the imaging of the eye includes: receiving, by the at least one processor, a segmentation measurement of one or more regions of the eye; and receiving, by the at least one processor, from a spectropolarimetric camera, a multi-dimensional spectropolarimetric measurement of the eye. In some embodiments, the present disclosure relates to a method, wherein classifying the patient into the at least one category includes: applying, by the at least one processor, one or more classification networks to the segmentation measurement and the multi-dimensional spectropolarimetric measurement to select a disease classification of the eye. In some embodiments, the present disclosure relates to a method, wherein the data from the imaging of the eye includes spectropolarimetric data packages including spectropolarimetric components relating to an anatomical location of the eye. In some embodiments, the present disclosure relates to a method, wherein the status of the patient with respect to the neurodegenerative disease includes a risk of the patient having or experiencing symptoms related to the neurodegenerative disease. In some embodiments, the present disclosure relates to a method, wherein the status of the patient with respect to the neurodegenerative disease includes a diagnosis of the patient as having the neurodegenerative disease. In some embodiments, the present disclosure relates to a method, wherein the status of the patient with respect to the neurodegenerative disease includes a progression of the neurodegenerative disease in the patient. In some embodiments, the present disclosure relates to a method, wherein the status of the patient with respect to the neurodegenerative disease includes a response of the patient to preventative interventions or treatment interventions. In some embodiments, the present disclosure relates to a method, wherein analyzing the data includes analyzing the spatial, spectral, and polarimetric data for the plurality of pixels with a regression model. In some embodiments, the present disclosure relates to a method, wherein classifying the patient into the at least one category includes classifying the patient based on a plurality of pathologies of the neurodegenerative disease. In some embodiments, the present disclosure relates to a method, wherein classifying the patient based on the plurality of pathologies includes classifying the patient based on a combined weighted score, scorecard, or probabilistic determination of each of the plurality of pathologies. In some embodiments, the present disclosure relates to a method, wherein analyzing the data from the imaging of the eye includes: performing semantic segmentation to identify different parts of the eye; and combining the semantic segmentation with the spatial, spectral, and polarimetric data for the plurality of pixels. In some embodiments, the present disclosure relates to a method, wherein analyzing the data further includes analyzing a relationship between the spatial, spectral, and polarimetric data for a first pixel with the spatial, spectral, and polarimetric data of two more adjacent pixels. In some embodiments, the present disclosure relates to a method, wherein analyzing the data includes analyzing the spatial, spectral, and polarimetric data for the plurality of pixels with an ensemble prediction model. In some embodiments, the present disclosure relates to a method, wherein each individual model of the ensemble prediction model is assigned a weight based on how significantly a prediction from each individual model correlates with amyloid or tau status of the patient. In some embodiments, the present disclosure relates to a method, wherein analyzing the data includes analyzing the spatial, spectral, and polarimetric data for the plurality of pixels with a convolutional neural network to generate a heatmap. In some embodiments, the present disclosure relates to a method, wherein analyzing the data includes calculating a quality assurance criterion for each of the plurality of pixels. In some embodiments, the present disclosure relates to a method, wherein analyzing the data includes evaluating the data for one or more biomarkers indicative of the neurodegenerative disease. In some embodiments, the present disclosure relates to a method, wherein the one or more biomarkers include Amyloid or Tau protein formations. In some embodiments, the present disclosure relates to a method, wherein the neurodegenerative disease is selected from the group consisting of Alzheimer's disease. Parkinson's disease. Amyotrophic Lateral Sclerosis. Multiple Sclerosis. Prion disease. Motor neurone diseases (MND), Huntington's disease (HD), Spinocerebellar ataxia (SCA), Spinal muscular atrophy (SMA), cerebral amyloid angiopathy (CAA).

The present disclosure relates to a system, including: a light source configured to illuminate an eye of a patient with light: an imaging device configured to receive light returned from the eye to generate a spectropolarimetric image of the eye, the spectropolarimetric image including a plurality of pixels and including, for each pixel, spatial, spectral, and polarimetric data; and a computing device configured to: receive the spectropolarimetric image: analyze the spatial, spectral, and polarimetric data for the plurality of pixels: based on the analyzing, classify the patient into at least one category of a plurality of categories, each category of the plurality of categories indicating a status with respect to a neurodegenerative disease that affects a central nervous system; and providing one or more of the at least one category as an output to indicate the status of the patient with respect to the neurodegenerative disease.

In some embodiments, the present disclosure relates to a system, further including generating the spatial, spectral, and polarimetric data synchronously as a single data package of spectropolarimetric data. In some embodiments, the present disclosure relates to a system, wherein: the spectropolarimetric image includes a multi-dimensional spectropolarimetric data package: analyzing the spatial, spectral, and polarimetric data includes applying one or more neural networks to each dimension of the multi-dimensional spectropolarimetric data package to generate a dimensional output for each dimension of the multi-dimensional spectropolarimetric data package; and classifying the patient into the at least one category includes combining each dimensional output of each dimension of the multi-dimensional spectropolarimetric data package. In some embodiments, the present disclosure relates to a system, wherein analyzing the spatial, spectral, and polarimetric data includes: receiving a segmentation measurement of one or more regions of the eye; and receiving, from the imaging device, a multi-dimensional spectropolarimetric measurement of the eye. In some embodiments, the present disclosure relates to a system, wherein classifying the patient into the at least one category includes applying one or more classification networks to the segmentation measurement and the multi-dimensional spectropolarimetric measurement to select a disease classification of the eye. In some embodiments, the present disclosure relates to a system, wherein spatial, spectral, and polarimetric data includes spectropolarimetric data packages including spectropolarimetric components relating to an anatomical location of the eye. In some embodiments, the present disclosure relates to a system, wherein the status of the patient with respect to the neurodegenerative disease includes a risk of the patient having or experiencing symptoms related to the neurodegenerative disease. In some embodiments, the present disclosure relates to a system, wherein the status of the patient with respect to the neurodegenerative disease includes a diagnosis of the patient as having the neurodegenerative disease. In some embodiments, the present disclosure relates to a system, wherein the status of the patient with respect to the neurodegenerative disease includes a progression of the neurodegenerative disease in the patient. In some embodiments, the present disclosure relates to a system, wherein the status of the patient with respect to the neurodegenerative disease includes a response of the patient to preventative interventions or treatment interventions. In some embodiments, the present disclosure relates to a system, wherein analyzing the spatial, spectral, and polarimetric data includes analyzing the spatial, spectral, and polarimetric data for the plurality of pixels with a regression model. In some embodiments, the present disclosure relates to a system, wherein classifying the patient into the at least one category includes classifying the patient based on a plurality of pathologies of the neurodegenerative disease. In some embodiments, the present disclosure relates to a system, wherein classifying the patient based on the plurality of pathologies includes classifying the patient based on a combined weighted score, scorecard, or probabilistic determination of each of the plurality of pathologies. In some embodiments, the present disclosure relates to a system, wherein analyzing the spatial, spectral, and polarimetric data includes: performing semantic segmentation to identify different parts of the eye; and combining the semantic segmentation with the spatial, spectral, and polarimetric data for the plurality of pixels. In some embodiments, the present disclosure relates to a system, wherein analyzing the spatial, spectral, and polarimetric data further includes analyzing a relationship between the spatial, spectral, and polarimetric data for a first pixel with the spatial, spectral, and polarimetric data of two more adjacent pixels. In some embodiments, the present disclosure relates to a system, wherein analyzing the spatial, spectral, and polarimetric data includes analyzing the spatial, spectral, and polarimetric data for the plurality of pixels with an ensemble prediction model. In some embodiments, the present disclosure relates to a system, wherein each individual model of the ensemble prediction model is assigned a weight based on how significantly a prediction from each individual model correlates with amyloid or tau status of the patient. In some embodiments, the present disclosure relates to a system, wherein analyzing the spatial, spectral, and polarimetric data includes analyzing the spatial, spectral, and polarimetric data for the plurality of pixels with a convolutional neural network to generate a heatmap. In some embodiments, the present disclosure relates to a system, wherein analyzing the spatial, spectral, and polarimetric data includes calculating a quality assurance criterion for each of the plurality of pixels. In some embodiments, the present disclosure relates to a system, wherein analyzing the spatial, spectral, and polarimetric data includes evaluating the data for one or more biomarkers indicative of the neurodegenerative disease. In some embodiments, the present disclosure relates to a system, wherein the one or more biomarkers include Amyloid or Tau protein formations. In some embodiments, the present disclosure relates to a system, wherein the neurodegenerative disease is selected from the group consisting of Alzheimer's disease. Parkinson's disease. Amyotrophic Lateral Sclerosis. Multiple Sclerosis. Prion disease. Motor neurone diseases (MND), Huntington's disease (HD), Spinocerebellar ataxia (SCA), Spinal muscular atrophy (SMA), cerebral amyloid angiopathy (CAA).

### BRIEF DESCRIPTION OF DRAWINGS

The present disclosure is further described in the detailed description which follows, in reference to the noted plurality of drawings through non-limiting examples of exemplary embodiments, in which like reference numerals represent similar parts throughout the several views of the drawings:

FIG. 1A shows a block diagram of the ocular imaging system with the light source, the polarization control, and the spectropolarimetric camera.

FIG. 1B shows a fundus camera.

FIG. 1C illustrates a view of the eye.

FIG. 1D shows the regions of the optic disc to be segmented.

FIG. 2A shows an illustration of the spectropolarimetric data package.

FIG. 2B-D show the spectropolarimetric data.

FIG. 3 shows a flowchart of a method for processing spectropolarimetric data packages.

FIG. 4 shows a plot of wavelength significance of a reflectance signal in the temporal zone with amyloid status.

FIG. 5 shows a flow chart of a method for wavelength feature identification.

FIG. 6A show an ensemble prediction model employing various features from spectropolarimetric imaging.

FIG. 6B show an ensemble prediction model employing various features from spectropolarimetric imaging.

FIG. 7A shows a plot of the probability amyloid status as calculated from the ensemble model.

FIG. 7B shows a plot of the receiver operator curve for classification of amyloid status from the predictive model.

FIG. 8 shows a method for processing spectropolarimetric data packages.

FIG. 9A shows a flowchart illustrating a method for analyzing vessels of the retina.

FIG. 9B shows representations of spectropolarimetric data packages relating to the method in FIG. 9A.

FIG. 9C shows a representation of segmentation of blood vessels in a retina using an AI.

FIG. 10 shows a block diagram of a computer-based system and platform.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Described herein are examples of techniques for neurodegenerative disease evaluation and/or diagnostics based on eye imaging. The techniques described herein can be used in some embodiments to generate an output indicating a status of a patient with respect to one or more medical conditions, such as a neurodegenerative disease that affects a central nervous system of a patient, where the patient can be an animal such as a human or non-human animal, a human or non-human vertebrate, or a human or non-human mammal. In generating an output indicating the status, a computing device can analyze data regarding an imaging of an eye of the patient. The data regarding the imaging can include data for a plurality of pixels of an image. A pixel can include spatial data resulting from the imaging which may depict one or more objects that were within field of view of an imaging device that acquired the image, and may, in some embodiments, additionally include spectropolarimetric data. Such spectropolarimetric data may include data for one or more spectropolarimetric bands captured using the imaging device, and such spectropolarimetric data may include polarization information captured using the imaging device. In some cases, in addition to or as an alternative to such spectropolarimetric data being captured, the data may be derived from the imaging of the eye of the patient. Analyzing the data can include analyzing the spectropolarimetric data (e.g., spatial, spectral, and polarimetric data) for the plurality of pixels. Additionally described herein are example techniques for classifying the patient into at least one category, each category indicating a status with respect to the neurodegenerative disease, based on an analysis of the spectropolarimetric data. In some cases, the at least one category can be provided as the output.

Conventionally, diagnosis or treatment of a neurodegenerative disease is imprecise, as a high-reliability determination would require an evaluation of substances present (or absent) within tissues of a patient's central nervous system. For example, such tissues may be evaluated to determine whether certain proteins are present or in what absolute or relative quantities. Review of a patient's central nervous system tissues, such as a biopsy or other review of brain tissues, however, is highly invasive and risks negative consequences for the patient, such as impact on the patient's neurological function. As such, often neurodegenerative diseases are not diagnosed using physical testing and instead an estimate is produced of whether the patient is experiencing a particular neurodegenerative condition. Such an estimate may be produced with cognitive assessments, such as through questions or completing tasks. In such cases, confirmation of the disease may be performed following the patient's death.

The inventors have also recognized that as an alternative to such estimates and to increase accuracy or reliability of diagnoses, functional/metabolic analyses have been developed that aim to identify presence or absence of proteins in the central nervous system without invasive procedures. While these conventional analyses can increase reliability of diagnosis, they also suffer from significant downsides. For example, such functional/metabolic analyses may rely on the patient being administered radioactive tracing substances, which interact with the patient's tissues and enable the testing system to detect the patient's proteins. For example, with positron emission tomography (PET), radiopharmaceuticals are administrated to trigger the patient's body to emit gamma rays, which are detected and used to build an image of the patient's central nervous system tissues. Such radioactive tracing substances can be harmful to patient's, leading patients, or clinicians to decline to use these tests. The tests are also costly, limiting their availability to patients and clinicians.

The inventors have thus recognized and appreciated that conventional approaches to diagnosing and treating neurodegenerative are not able to provide a convenient, low-risk and low-intervention test of a patient to yield information regarding status with respect to neurodegenerative diseases.

As the eye is an extension of the central nervous system, linked by the optic nerve directly to the brain, many neurogenerative or neurological conditions or diseases affecting the brain can manifest in the eye, such as protein accumulation, changes to the structure of retinal layers, and other changes in chemical composition, structure, and function. For example, proteins produced in a patient's brain can migrate from the brain to the fundus of the eye. In another example, proteins produced in the brain as part of Alzheimer's disease progression such as beta amyloid and tau migrate from the brain to the fundus of the eye. In individuals with Alzheimer's disease, both the amyloid and tau levels in the brain are elevated prior to the onset of symptoms. The levels of amyloid and tau are correlated, in that subjects who develop AD tend to have biomarker evidence of elevated amyloid deposition biomarkers (which is detected via abnormal amyloid PET scan or low CSF Ab42 or Ab42/Ab40) ratio) as the first identifiable evidence of abnormality, followed by biomarker evidence of pathologic tau (which is detected via CSF phosphorylated tau, and Tau PET). This may be due to amyloid pathology inducing changes in soluble tau release, leading to tau aggregation later. Examples of neurological diseases that affect the eye include Alzheimer's disease. Parkinson's disease. Amyotrophic Lateral Sclerosis. Multiple Sclerosis. Prion disease. Motor neuron diseases (MND), Huntington's disease (HD), Spinocerebellar ataxia (SCA), Spinal muscular atrophy (SMA), and cerebral amyloid angiopathy (CAA). By examining the eye to identify physical changes, these and other neurological diseases can be identified early on to improve health outcomes.

The inventors have recognized and appreciated that, given this direct connection via neurological tissue, a patient's eye may contain indications of a condition of the patient's brain, and determinations regarding the patient's eye may be used to infer a condition of the patient's brain. The eye can be examined using a variety of non-invasive light-based techniques to identify biomarkers because conditions affecting the optic nerve and retina can result in changes that induce different polarization changes in reflected light as a function of wavelength of the light. The detection of biomarkers in the eye can be indicative of the presence or absence of proteins in the brain or the central nervous system and corresponding risk of developing diseases. By examining the eye to identify physical changes, these and other neurological diseases can be identified early on to improve health outcomes.

Described below are techniques for analyzing the eye to detect one or more biomarkers of disease, which might be evaluated to determine a status of a patient with respect to a disease, such as a diagnosis. In some embodiments, the system and methods of the present disclosure capture images comprising spectra components to identify biomarkers indicative of disease. The inventors have recognized and appreciated that biomarkers may be detectable from spectropolarimetric data packages captured of the patient's eye, such as when the eye is illuminated with light (e.g., visible light of one or more colors, including white light, and/or non-visible light of one or more ranges) and light reflected or otherwise output from the eye is captured with imaging equipment.

In some embodiments, images or data packages comprising spectropolarimetric data from illumination and imaging of the eye may be obtained in a non-invasive manner for a patient and may present relatively low risk of injury for the patient, or lower risk than invasive techniques. By analyzing the data related to the images or data packages, a testing system may determine whether one or more biomarkers are present or absent in the patient's eye and/or determine absolute or relative amounts of the biomarker(s) in the patient's eye. A testing system may then use this information to determine whether the biomarker(s) are present in the patient's brain and/or the absolute or relative amount(s) of the biomarker(s) in the patient's brain, and/or a determination of the patient's status with respect to one or more neurodegenerative diseases. Accordingly, by making determinations regarding proteins or protein levels (or other biomarkers) for a patient's eye using images or data packages of the eye, a patient's status regarding one or more neurodegenerative diseases may be obtained. For example, the ability to measure biomarkers in the images according to the present disclosure enables a measure of biomarkers in ocular tissues, such as the retina and optic disc, and use of these measures as a proxy for the levels of biomarkers in the brain to detect a neurological disease such as Alzheimer's.

Described herein are techniques for analyzing data related to an image of a patient's eye to determine the patient's status with respect to neurodegenerative diseases like Alzheimer's Disease (AD). More particularly, techniques described herein analyze data related to an image (e.g., a data package determined based at least in part from the image) of a patient's eye to identify whether one or more proteins or other biomarkers are present in the eye and make determinations of the patient's status with respect to one or more neurodegenerative diseases based on such presence or absence of the biomarkers. In some embodiments, techniques described herein may be used to estimate an amount of one or more biomarkers present in the patient's eye based on the data related to the image of the eye. In some embodiments, techniques described herein may be used to estimate relative amounts of one or more biomarkers present in the patient's eye based on the data related to the image of the eye. The patient's status with respect to a neurodegenerative disease that is determined based on the analysis of the data may include determining the patient's risk of having or experiencing symptoms related to the neurodegenerative disease, diagnosing the patient as having the neurodegenerative disease, monitoring the patient's progression with respect to the neurodegenerative disease, and/or monitoring the patient's response to preventative interventions or treatment interventions to mitigate the patient's risk of developing the neurodegenerative disease or experiencing symptoms of the neurodegenerative disease.

In some embodiments, the systems and methods of the present disclosure can be used to detect, from images of an eye of the patient and/or data packages determined using such an image or images, various disease biomarkers, such as, for example. Tau neurofibrillary tangles. Amyloid Beta deposits, soluble Amyloid Beta aggregates, or Amyloid precursor protein in the brain or the central nervous system. In some embodiments, the systems and method of the present disclosure may detect biomarkers indicative of tau pathologies or tauopathies, including, without limitation, total (T-tau), Tau PET, and phosphorylated tau (P-tau), In some embodiments, the biomarkers indicative of a Tauopathy include, but are not limited to, phosphorylated paired helical filament tau (pTau). Early Tau phosphorylation. Late Tau phosphorylation, pTau181, pTau217, pTau231, total Tau. Plasma AB 42/40. Neurofibrillary tangles (NFTs) and aggregation of misfolded tau protein. In some embodiments, neurofilament light protein (NFL), neurofilaments (NFs) or abnormal/elevated neurofilament light protein (NFL) concentration can be detected. In some embodiments, surrogate markers of a neurodegenerative disorder or neuronal injury can be detected, for example, retinal and optic nerve volume loss or other changes, degeneration within the neurosensory retina, and optic disc axonal injury. In some embodiments, an inflammatory response or neuroinflammation may be detected and may be indicative of neurological disease. In some embodiments, such inflammatory response may be detected in the retinal tissue. Examples of such responses include, but are not limited to, retinal microglia activation, degenerating ganglion cells (ganglion neuron degeneration) or astrocyte activation. Other protein aggregates or biomarkers useful in the methods and systems of the present disclosure include alpha synuclein and TDP43 (TAR DNA binding protein-43) and others described, for example, in Biomarkers for tau pathology (Molecular and Cellular Neuroscience. Volume 97. June 2019. Pages 18-33), incorporated herein by reference in its entirety. In some embodiments, the systems and methods of the present disclosure can be used to detect the presence or absence of protein aggregates or other biomarkers indicative of one or more neurological diseases in the patient's eye tissue, brain tissue, tissues of the central nervous system, peripheral nervous system, or in the cerebrospinal fluid (CSF) or any other tissue where such formations or their biomarkers occur. In some embodiments, the systems and methods of the present disclosure detect protein aggregates or other biomarkers indicative of one or more neurological diseases without using a dye or ligand. In some embodiments, dyes or ligands may be used to assist the presently disclosed methods and systems. In some embodiments, the results of the optical tests can be confirmed using an anatomic MRI. FDG PET, plasma test, and/or CSF total Tau.

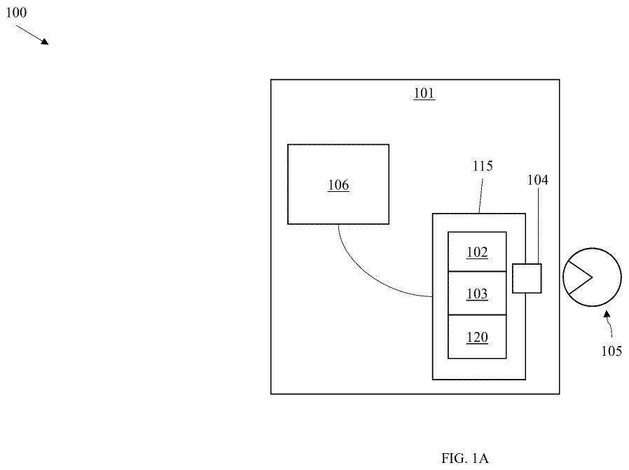

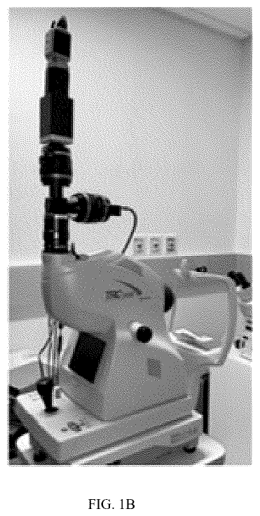

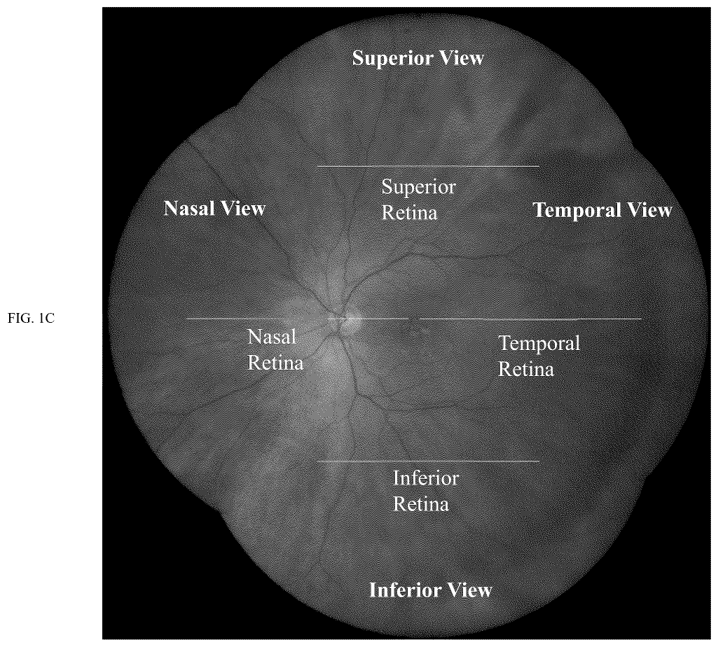

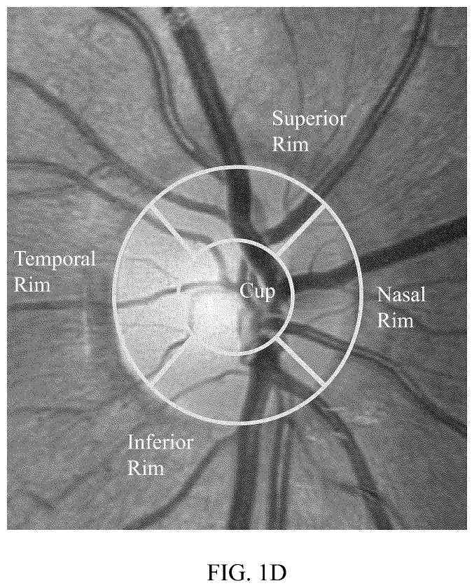

Referring now to FIG. 1A, various imaging systems may be employed to gather spectropolarimetric data. By way of a non-limiting, example. FIG. 1A shows an ocular imaging system 100 for capturing one or more scans of the eye 105 for pathology detection or for diagnosing neurological diseases, such as Alzheimer's disease. The ocular imaging system 100 can be a ophthalmic spectropolarimetric system 101. The ophthalmic spectropolarimetric system 101 can generate one or more data packages of the eye 105, receive the one or more spectropolarimetric data packages of the eye 105, evaluate the one or more spectropolarimetric data packages, and identify one or more biomarkers indicative of a neurodegenerative pathology.

In some embodiments, the ophthalmic spectropolarimetric system 101 includes a spectropolarimetric camera 102, a light source 103 for generating light to pass through one or more optical elements 104 to illuminate an eye 105, the one or more optical elements 104 configured to pass light to the eye 105 and receive the light reflected or otherwise returned by the eye 105, and a polarizer 120 configured to polarize the light. The spectropolarimetric camera 102 can include one or more imaging sensors for generating the image of the eye 105 based on the light reflected or emitted by the eye 105. The spectropolarimetric camera 102 can include one or more imaging sensors for generating the spectropolarimetric data package of the eye 105 based on the light reflected by or emitted by the eye 105. In some embodiments, the ophthalmic spectropolarimetric system 101 includes a computing device 106 configured to receive the one or more spectropolarimetric data packages, evaluate the spectropolarimetric data packages, identify one or more biomarkers indicative of a neurodegenerative disease, and determine status with respect to (e.g., presence or risk of) one or more neurodegenerative conditions.

In some embodiments, the spectropolarimetric camera 102, the light source 103, and the polarizer 120 can be in communication with a computing device 106 for obtaining and analyzing the spectropolarimetric data packages. In some embodiments, the spectropolarimetric data package can be generated by the spectropolarimetric camera 102 for analysis by the computing device 106. In some embodiments, the ophthalmic spectropolarimetric system 101 can generate one or more spectropolarimetric data packages of the eye 105, receive the one or more spectropolarimetric data packages of the eye 105, evaluate the one or more spectropolarimetric data packages, determine whether evidence of or information regarding one or more biomarkers indicative of a neurodegenerative pathology is present in the spectropolarimetric data package, and determine status with respect to (e.g., presence or risk of) a neurodegenerative condition based on the biomarkers identified in the spectropolarimetric data package. In some embodiments, the computing device 106 can identify the pathologies by analyzing the spectropolarimetric data package generated by the spectropolarimetric camera 102 of the eye 105. In some embodiments, the computing device 106 can identify the pathologies by analyzing the spectropolarimetric data package derived from the image during preprocessing of the spectropolarimetric data package. In some embodiments, the ophthalmic spectropolarimetric system 101 can calibrate the generation of the spectropolarimetric data packages and edit the spectropolarimetric data packages to remove artifacts and prepare spectropolarimetric data packages for diagnostic imagery analysis.

In some embodiments, the ophthalmic spectropolarimetric system 101 is a non-invasive ocular light-based system for detecting neurodegenerative disease-associated pathologies in the eye 105. In some embodiments, the ophthalmic spectropolarimetric system 101 can be used to generate spectropolarimetric data packages of the eye 105 by providing broadband illumination and imaging optics, including an integrated or external camera to capture the spectropolarimetric data packages of the fundus of the eye 105. In some embodiments, the ophthalmic spectropolarimetric system 101 can provide illumination and spectropolarimetric data packages of the posterior of the eye 105 (using an internal integrated camera).

The ophthalmic spectropolarimetric system 101 can be a light-based tool that provides an accessible and non-invasive procedure for identifying, diagnosing, and tracking treatment and intervention efficacy of populations at-risk for neurological diseases. The ophthalmic spectropolarimetric system 101 can be used for optical examination and imaging of part of the fundus, such as the retina to look for signs of AD-associated pathologies in the subject's eye 105 tissue, brain tissue, tissues of the central nervous system, in cerebrospinal fluid (CSF) or any other tissue where such formations or their biomarkers occur. In some embodiments, dyes or ligands may be used to assist with imaging the tissues. In some embodiments, the results of the optical tests can be confirmed using an anatomic MRI, FDG PET, plasma test, and/or CSF total Tau.

Various imaging systems may be employed to gather spectropolarimetric data. The ophthalmic spectropolarimetric system 101 of the present disclosure can be presented as a stand-alone imaging system. In some embodiments, the ophthalmic spectropolarimetric system 101 of the present disclosure can be incorporated into a fundus camera or a similar ophthalmology examination device.

By way of a non-limiting example. FIG. 1B shows a fundus camera for use with the ophthalmic spectropolarimetric system 101 for capturing one or more scans of the eye 105 and generating a spectropolarimetric data package for pathology detection or for diagnosing disease, such as Alzheimer's disease or any other disease mentioned herein. In some embodiments, the ocular imaging systems of the present disclosure may be presented as a stand-alone imaging system. In some embodiments, the ocular imaging systems of the present disclosure may be incorporated into a fundus camera or a similar ophthalmology examination device. In some embodiments, the ophthalmic spectropolarimetric system 101 described herein can generate such a spectropolarimetric data package by using the spectropolarimetric camera 102 with the fundus camera. In some embodiments, the fundus camera is a Topcon NW8, EX, or DX. In some embodiments, the fundus camera includes an external camera port. An operator can adjust the focal length and illumination power of the ophthalmic fundus camera while capturing images of the eye 105. For example, the operator can use one or more knobs to adjust position of the optics and thus adjust the focal length, and/or adjust the illumination power of a light source. The knob(s) may directly control position of the optics, such that as the knob(s) is turned the optics move (e.g., through action of one or more gears or other mechanical elements connected between the knob(s) and the optic(s), or through other mechanisms), and the optics may be continuously adjustable. By being continuously adjustable, the optics may be positioned at any location along a movement path of the optics, rather than only be positioned at discrete positions along the movement path. In some embodiments, the fundus camera may not be configurable to determine, store, or output the position of the optics or the focal length of the optics.

Various views of the eye 105 can be acquired as shown in FIG. 1C. In some embodiments, the ocular imaging systems can be used to image the fundus of the eye 105 by providing broadband illumination and imaging optics, including an integrated or external camera to capture the image of the fundus of the eye 105. In some embodiments, the ocular imaging systems can provide illumination and image the posterior of the eye 105 (using an internal integrated camera).

As shown in FIG. 1C, the images can be regionally segmented to identify pixels in the various components of the eye 105, including the optic disc (nerve head), retina, and fovea. The ophthalmic spectropolarimetric system 101 can identify or determine the existence of one or more AD-associated pathologies, including, but not limited to, protein aggregates, where the protein aggregates can include at least one of: Tau neurofibrillary tangles, Amyloid Beta deposits, soluble Amyloid Beta aggregates, or Amyloid precursor protein. In some embodiments, the ophthalmic spectropolarimetric system 101 can use a first imaging modality to identify the locations of blood vessels in the eye 105 (e.g., based on spatial components in an image and/or by detecting blood flow from the image). In some embodiments, the ophthalmic spectropolarimetric system 101 can use a second imaging modality to analyze the spectropolarimetric components of the blood vessels where the neurological disorders or pathologies may be more likely to be evident.

In some embodiments, the ophthalmic spectropolarimetric system 101 can segment the regions within the optic disc to identify more specific components, including a temporal rim, nasal rim, inferior rim, superior rim, and cup regions as shown in FIG. 1D. In some embodiments, the computing device 106 can perform the segmentation with an automated segmentation algorithm.

Referring to FIG. 1A, the light source 103 can be configured to illuminate the eye 105. In some embodiments, the light source 103 may be a broadband light source 103, which emits a wide spectrum of light (e.g., UV, visible, near infrared, and/or infrared wavelength ranges). In some embodiments, the light source 103 may be a narrow band light source 103 which emits a narrow spectrum or single wavelength of light. In some embodiments, the light source 103 may emit a single continuous spectrum of light. In some embodiments, the light source 103 may emit a plurality of discontinuous spectra. In some embodiments, the light source 103 may emit light with a constant wavelength band or intensity. In some embodiments, the wavelengths composition of the light source and its intensity may be adjustable. In some embodiments, the light source 103 is configured to emit light only at wavelengths relevant for calculating the metrics indicative of systemic and localized diseases (e.g., Age Related Macular Degeneration, Retinopathy) and/or a metabolic state (e.g., oxygenation, blood circulation, bleaching of photoreceptors). In some embodiments, the light source 103 may comprises one or more super luminescent diodes (SLEDs), light emitting diodes (LEDs), xenon flashlight source, laser, or light bulbs, a xenon lamp, a mercury lamp, or any other illuminator and light emitting elements. The light source 103 can include a single source of light or a combination of multiple sources of light of the same or different types described above.

In some embodiments, the light source 103 generates light having a known or predetermined polarization. In some embodiments, the light source 103 may emit light circularly polarized, with one or more known polarization components (e.g., known spatial characteristics, frequencies, wavelengths, phases, and polarization states). In some embodiments, the light source 103 may emit light with a random polarization (e.g., light that has a random mixture of waves having different spatial characteristics, frequencies, wavelengths, phases, and polarization states).

The polarizer 120 can comprise a polarization filter array comprising one or more polarization filters that transmit light waves of a specific polarization while blocking light waves of other polarizations. In some embodiments, the polarizer 120 can be a mechanical, electromechanical, or electrooptical device that rotates the transmitted polarization light using a mechanical, electromechanical, or electrooptical driven mechanism (e.g., Pockels cells, rotating polarizers, liquid crystal device etc.). In some embodiments, the polarizer 120 can provide linear, elliptical, or circular polarization. The polarizer 120 can reduce reflections, reduce atmospheric haze, and increase color saturation in the spectropolarimetric data packages. The polarizer 120 can be an array of polarization filters used to capture and measure different polarizations of incoming light on different pixels at the same time. The filter can provide polarization states at any one or more angles, such as 0, −45, 45, and 90 degrees. In some embodiments, the polarizer 120 can restrict the polarization of light that illuminates the eye 105 at any given time. In some embodiments, the polarizer 120 is an array of polarization filters each corresponding with one or more pixels of the spectropolarimetric camera 102. The polarizer 120 can be used to capture and measure different polarizations of incoming light sequentially by allowing light through the polarizer 120. In some embodiments, the polarizer 120 may be combined with or otherwise work in combination with a spectropolarimetric filter array comprising one or more spectropolarimetric filters to limit the wavelengths of light received by the spectropolarimetric camera 102 to the wavelengths relevant for calculating the metrics indicative of disease state. In some embodiments, the light source 103 includes the polarizer 120 to control or restrict the polarization of light that illuminates the eye 105. In some embodiments, the polarizer 120 controls or restricts the polarization of light reflected, emitted, or returned from the eye 105 that is received by the spectropolarimetric camera 102.

In some embodiments, the polarizer 120 can be placed between the light source 103 and the eye 105. In some embodiments, the polarizer 120 may be used to polarize the illumination source 103. In some embodiments, the polarizer 120 can be used to polarize the light collected by the spectropolarimetric camera 102 from the eye 105. In some embodiments, the polarizer 120 can be placed between the eye 105 and the spectropolarimetric camera 102. In some embodiments, the polarizer 120 can be placed between both the light source 103 and the eye 105, and another polarizer 120 can be placed between the eye 105 and the spectropolarimetric camera 102. In some embodiments, the polarizer 120 can be integrated with the light source 103 or with the spectropolarimetric camera 102 and in some embodiments, it can be separate. In some embodiments, the polarizer 120 may be placed both between the light source 103 and the eye 105, and between the eye 105 and the spectropolarimetric camera 102.

The spectropolarimetric camera 102 can be a device or sensor configured to receive light returned from the eye 105. In some embodiments, the spectropolarimetric camera 102 can generate one or more spectropolarimetric data packages based on the light reflected from the eye. In some embodiments, the spectropolarimetric camera 102 may capture spectropolarimetric data that comprises spectral, spatial, and polarimetric components from which one or more spectropolarimetric data packages can be constructed. In some embodiments, the spectropolarimetric camera 102 may capture spectropolarimetric data that comprises spectral, spatial, and polarimetric components of the same and different part of the object.

The spectropolarimetric camera 102 may be any sensor or camera configured to collect and record spectropolarimetric data packages from the eye 105 or, in particular, the fundus of the eye 105. Various embodiments of such cameras are disclosed in co-pending and co-owned patent applications (for example, U.S. 63/425,155 filed on Nov. 14, 2022), which are incorporated herein by reference in their entireties.

The light source 103 may direct light toward the eye 105 and the spectropolarimetric camera 102 may be configured to collect and record light reflected, emitted or otherwise returned from by the eye 105. In some embodiments, the light source 103 can direct the light toward the eye 105 with the same optical assembly (including the one or more optical components 104) configured to collect light from the eye 105. In some embodiments, the light source 103 may direct light toward the eye 105 through a different optical path. The spectropolarimetric camera 102 can generate spectropolarimetric data packages of the eye 105 from light emitted from the light source 103, reflected, emitted, or otherwise returned by the eye 105, and received by the spectropolarimetric camera 102. The spectropolarimetric camera 102 can produce a measurement or the spectropolarimetric image of the eye 105 or any single component of the eye 105.

In some embodiments, the spectropolarimetric camera 102 can be a spectropolarimetric imaging sensor that can produce or generate the spectropolarimetric data packages. In some embodiments the light sensible sensor can be single pixels, a line of pixels or a matrix of pixels. In some embodiments, in addition to the spectropolarimetric camera 102, optical coherence tomography (OCT) or confocal scanning laser ophthalmoscopy (SLO) can be used to enhance and collect the spectropolarimetric data packages. In some embodiments, one or more single photon avalance detectors (SPADs), photomultiplier tubes (PMTs), or other photon sensing devices can also be used. In some embodiments, the spectropolarimetric camera 102 includes a spectropolarimetric sensor. In some embodiments, the spectropolarimetric sensor can be a snapshot spectropolarimetric sensor, push broom spectropolarimetric camera, whiskbroom spectropolarimetric camera, staring spectropolarimetric camera. In some embodiments, the spectropolarimetric sensor can be a spectropolarimetric sensor, multispectral sensor, monochrome sensor, or an RGB sensor. In some embodiments, the spectropolarimetric sensor may be a Fourier transform spectrometer used with a broadband light source. In some embodiments, any imaging system that allows for the collection of spectropolarimetric data packages may be used. In some embodiments, the spectropolarimetric sensor may be a monochromatic sensor or other imaging device used with a tunable light source, and/or multiple light sources of different wavelengths, and/or a broadband light source with spectropolarimetric filters to generate the spectropolarimetric components. In some embodiments, the spectropolarimetric sampling can be performed in the illumination optical path and/or in the detection optical path. In some embodiments, the spectropolarimetric sampling can be performed using optomechanical (e.g., filter wheel), electro-optical (e.g., electro optical filter, liquid crystal), acusto-optical (e.g., acusto-optical filters) tunable filters device. The spectropolarimetric camera 102 can be any optical assembly that allows the recording of an image of an object, a scene or a sample. The spectropolarimetric camera 102 can be a microscope (e.g., wide field, confocal), or optical coherence tomography system which contain spectropolarimetric cameras 102 (e.g., a camera) configured to receive the spectropolarimetric data packages and communicate with a computer to transmit the spectropolarimetric data packages for analysis. The spectropolarimetric camera 102 can include one or more objective lenses and a camera sensor.

In some embodiments, a plurality of spectropolarimetric cameras 102 can be used to capture spectropolarimetric data packages at the same time or in sequence. In some embodiments, the plurality of spectropolarimetric cameras 102 capture the spectropolarimetric data packages with different magnification, field of view, spatial resolution, and/or spectropolarimetric resolution by using different spectropolarimetric cameras 102. In some embodiments, a first spectropolarimetric camera 102 could be coupled with the ophthalmic spectropolarimetric system 101 to produce a first spectropolarimetric data package and then a second spectropolarimetric camera 102 could produce a second spectropolarimetric data package. In some embodiments, the plurality of spectropolarimetric cameras 102 capture the spectropolarimetric data packages so that the spectropolarimetric data package from a first spectropolarimetric camera 102 can be analyzed to identify spatial, spectral, or polarization components and determine which second spectropolarimetric camera 102 should be used and/or which locations or portions of the eye 105 to measure with a second spectropolarimetric camera 102. In some cases, instead of using a second spectropolarimetric camera 102, the first spectropolarimetric camera 102 could be used with different settings (e.g., magnification or field of view) to capture a second spectropolarimetric data package of the eye 105 with different spatial, spectral, or polarization components and resolution.

In some embodiments, the spectropolarimetric camera 102 comprises a scanning point spectrometer that generates the spectropolarimetric data packages in two dimensions. In some embodiments, the scanning spectrometer that can produce the spectropolarimetric data packages with both high spatial resolution and high spectropolarimetric resolution with scanning optics and software. In some embodiments, the spectropolarimetric camera 102 comprises a line spectrometer that generates the spectropolarimetric data packages in one dimension (also referred to as a whisk broom imager). In some embodiments, the spectropolarimetric camera 102 comprises a matrix spectrometer that generates the spectropolarimetric images in two dimensions (also referred to as a push broom imager). In some embodiments, a line spectrometer can be used to produce a one-dimensional spectropolarimetric data package with a polarization data package at each wavelength for each pixel along a line without scanning (e.g., 1×N), and a point spectrometer can produce a point image (e.g., 1×1) without scanning. In some embodiments, a line spectrometer or point spectrometer can be used to produce higher dimensional spectropolarimetric data packages with spatial, spectral and or polarization scanning. In some embodiments, the imaging techniques allow the production of three-dimensional spectropolarimetric data packages in which a spectropolarimetric data package is produced for each pixel in a three-dimensional volume.

In some embodiments, the spectropolarimetric camera 102, the light source 103, and the polarizer 120 can be placed inside a housing 115 with the one or more optical elements 104 configured to direct light from the light source 103 to the eye 105, and direct light reflected, emitted, or returned from the eye 105 to the spectropolarimetric camera 102. In some embodiments, the element(s) of the system 101 that performs the evaluation of the packages, identification of the biomarkers, and determination of presence or risk of a disease may be integrated with the spectropolarimetric camera 102 in the same housing 115. In some embodiments, the spectropolarimetric camera 102, the light source 103, the computing device 106, and the polarizer 120 can be placed inside the housing 115. In some embodiments, the housing 115 can be a fundus camera. In some embodiments, the spectropolarimetric camera 102, light source 103, or polarizer 120 can be integrated into the housing 115. In some embodiments, the spectropolarimetric camera 102 can be in the form of a stand-alone device or a sensor configured to be attached to the housing 115. In some embodiments, the light source 103 and/or the polarizer 120 are attached to the ophthalmic spectropolarimetric system 101. In some embodiments, the light source 103, the spectropolarimetric camera 102, and/or the polarizer 120 are separate from the housing 115. In some embodiments, the system 101 may further include an array of one or more spectropolarimetric filters, either integrated with the polarizer 120 or as a standalone component of the ophthalmic spectropolarimetric system 101. In some embodiments, the element(s) that perform these functions may be separate from the spectropolarimetric camera 102, such as in a computing device 106 that is outside the housing 115.

In some embodiments, the ophthalmic spectropolarimetric system 101 includes a wavelength calibration source that emits narrowband light at one or more specific known wavelengths. The wavelength calibration source can be located within the housing 115 or placed externally to the housing 115. In some embodiments, the wavelength calibration source can be coupled to the light source 103. In some embodiments, the wavelength calibration source can be next to the light source 103. The computing device 106 can receive a wavelength calibration signal from the spectropolarimetric cameras 102 that capture the light emitted by the wavelength calibration source. The computing device 106 can calculate a pixel to wavelength conversion for spectropolarimetric data packages from the corresponding wavelength calibration signal. Since the wavelength calibration source emits light at specific known wavelengths, the computing device 106 can assign the known wavelengths to the pixels on which the light falls. The computing device 106 can interpolate/extrapolate based on the known wavelengths to assign wavelength values to other pixels.

In some embodiments, the computing device 106 can be configured to obtain, request, or receive a retinal image mosaic comprising the spectropolarimetric data packages of the eye 105. In some embodiments, the computing device 106 can analyze the one or more spectropolarimetric data packages to identify biomarkers indicative of a neurodegenerative pathology. In some embodiments, the computing device 106 can generate a digital representation indicative of a presence or absence of the biomarkers in the one or more regions of the eye 105.

The computing device 106 can perform wavelength calibration using a previously acquired spectrum of a mercury or mercury-argon lamp, or other light source 103 with well-defined spectropolarimetric characteristics. The positions of wavelengths of the peaks in a mercury spectrum have well-defined characterized wavelengths via NIST or other standards. The computing device 106 can compare the known wavelengths and the position of the peaks in the mercury or mercury-argon lamp spectrum with the spectrum measured by the spectropolarimetric camera 102 and the pixels where those wavelengths and the position of those peaks appear in the measured spectrum. The computing device 106 can use the comparison to allow for a pixel to wavelength mapping to be calculated for the spectropolarimetric data package and the wavelengths of light in subsequent spectropolarimetric data packages to be known. The pixels in the spectropolarimetric data packages where the peaks of the mercury lamp are measured can be assigned to the known wavelengths of those peaks. By noting the pixels where each of the known mercury or mercury-argon lamp peaks is measured, the computing device 106 can calculate an interpolation function to map each spatial pixel to a wavelength value. This interpolation function can be used to correctly assign the wavelength values of each pixel in subsequent spectropolarimetric data packages.

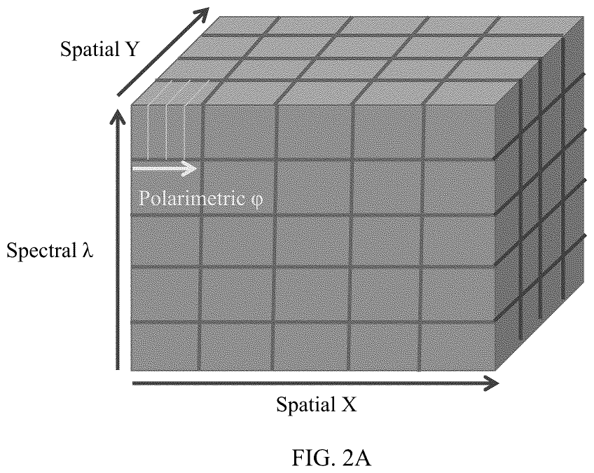

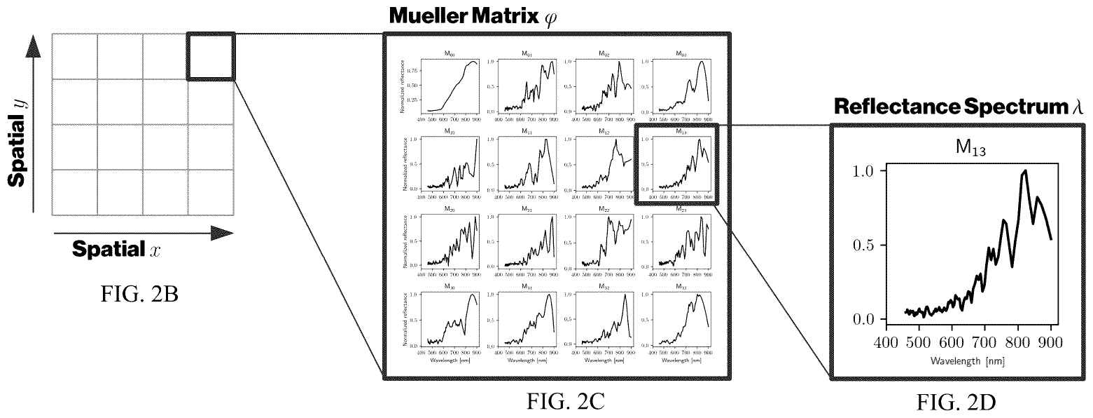

Referring now to FIGS. 2A and 2B, in some embodiments, pathologies can be identified by analyzing the spectropolarimetric data package that includes the image of the eye 105 or that can be derived from the image of the eye 105 during preprocessing of the image. In some embodiments, the spectropolarimetric data package comprises spectropolarimetric components obtained from polarized light reflected or otherwise returned from the eye 105. In some embodiments, the spectropolarimetric data package of the eye 105 can be generated by the spectropolarimetric camera 102 for analysis by the computing device 106.

Referring now to FIG. 2A, in some embodiments, the spectropolarimetric data package can be visualized as a spectropolarimetric 4-D data set. The spectropolarimetric data package can include four-dimensional data or images (4-D image). In some embodiments, the spectropolarimetric data package includes data elements of (X, Y, λ, φ). In some embodiments, the spectropolarimetric data package (which can include spectropolarimetric components, spectral-spatio-spectral components, spatial-spectral components, or spatial spectropolarimetric components) can include a spatial X component, a spatial Y component, a spectral λ component of wavelength, and a polarimetric φ component.

Now referring to FIG. 2B, in some embodiments, the spectropolarimetric data package can identify each pixel on a x-y grid that encodes both spectrum (λ) and polarization (φ) parameters. In some embodiments, the spectropolarimetric data package can comprise a 2-dimensional spatial array in which each pixel can be associated with 2 or more spectropolarimetric components measured at 2 or more different wavelengths. The spectropolarimetric data packages described herein can include “pixels” that extend the classic definition of a pixel from a colored point in an image to a point that has, in itself, two dimensions of data (spectral and polarimetric). Therefore, the spectropolarimetric data packages can include pixels that each include spatial, spectral, and polarimetric data.

Now referring to FIG. 2C, the spectropolarimetric components may be represented in a 4×4 Mueller matrix that describes the reflectance of the eye 105 at various wavelengths. The input vector can be the incident light directed at the eye 105 from the light source 103 and the output vector can be the light reflected or otherwise returned from the eye 105 to the spectropolarimetric camera 102. In some embodiments, the vectors are represented as a 4-element Stokes vector, or as other representations of the polarization of the incident and/or reflected light. Since the polarization state of the input and output light can be defined by four element vectors, the polarization components can be encoded on a 16-element Mueller Matrix with four polarization angles (for example, 0, −45, 45, 90) for both polarization state generator (PSG) (input light) and polarization state analyzer (PSA) (output light). Each element of the Mueller Matrix can indicate the reflectance of the eye 105 at various wavelengths at a specific polarization ratio of the input and output light. For example, the Mueller Matrix element Moo corresponds to hyperspectral imaging without polarization. As shown in FIG. 2D, the Mueller matrix element Mis indicates a reflectance spectrum λ at a particular ratio of polarization of input light and output light.

In some embodiments, the spectropolarimetric data package is a data package that comprises spatial and polarimetric components without spectral components. In some embodiments, the spectropolarimetric data package can be a 2-D spatial image with a polarization measurement of the light at two or more wavelengths for each image pixel (or a three-dimensional spatial image with a polarization measurement of the light at two or more wavelengths for each image voxel). In some embodiments, the data package comprises spectropolarimetric components obtained from polarized light reflected from the eye 105. In some embodiments, the polarimetric component can be at polarization angles such as 0, −45, 45, or 90.

In some embodiments, the spectropolarimetric data package can include a 3-D spatial array generated by using a volumetric imaging technique such as optical coherence tomography (OCT). Each element in the spatial array may have arrays of wavelength and polarization values associated with it. In some embodiments, the spectropolarimetric data package can include dimensionality based on plenoptic (light field) data packages or time-varying dynamic data packages.

The spectropolarimetric data packages generated herein can allow for accurate patient and pathology classification. Particularly, all four dimensions of the spectropolarimetric data package (a spatial X component, a spatial Y component, a spectral λ component of wavelength, and a polarimetric φ component) can be evaluated at the same time, revealing hyper-patterns in the hyper-space of the generated spectropolarimetric images or data packages. Such synchronous evaluation of the four dimensions can reveal more information for patient or pathology classification than individually collecting and analyzing spatial, spectral, spatial-spectral, and polarimetric data on a patient's eye.

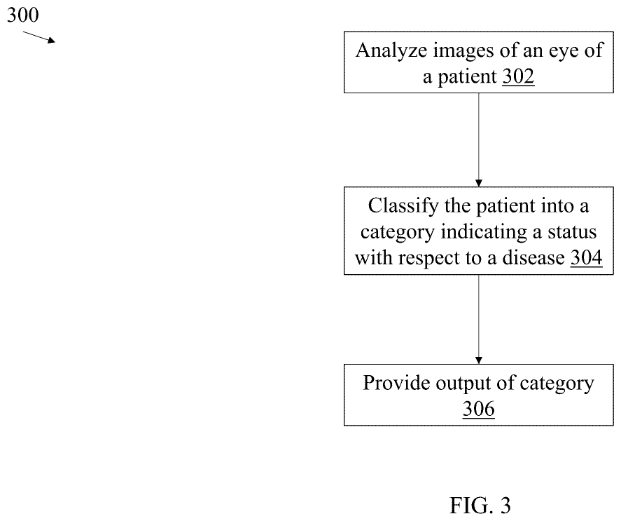

FIG. 3 illustrates a method 300 for processing spectropolarimetric data packages that include spectropolarimetric components. In some embodiments, the method 300 may be performed by the computing device 106. In some embodiments, the computing device 106 can receive spectropolarimetric data packages using an ocular imaging system 101 (as for example, shown in FIG. 1A). In some embodiments, the ocular imaging system 101 can include a light source 103 for illuminating the eye 105. In some embodiments, the ocular imaging system 101 can include a spectropolarimetric camera 102 configured to receive light reflected or otherwise returned from the eye 105 and capable of capturing spectropolarimetric data packages. In some embodiments, the ocular imaging system 101 can include a computing device 106 in communication with the spectropolarimetric camera 102 to receive and evaluate the spectropolarimetric data packages.

At step 302, the ocular imaging system 101 analyzes the images of one or more regions of the eye 105. The computing device 106 can cause the light source 103 to illuminate the eye 105 with light. The computing device 106 can receive or maintain the images generated by the spectropolarimetric camera 102 of the ocular imaging system 101. The computing device 106 can cause the spectropolarimetric camera 102 to generate one or more images from light received from the eye 105. The computing device 106 can evaluate the images to identify one or more biomarkers indicative of a neurodegenerative disease.

The computing device 106 can receive and analyze spectropolarimetric data packages generated by the spectropolarimetric camera 102. In some embodiments, the computing device 106 can receive the one or more spectropolarimetric data packages from the spectropolarimetric camera 102. The spectropolarimetric camera 102 can be coupled to the computing device 106. In some embodiments, the outputs of the spectropolarimetric camera 102 can be coupled to the computing device 106, such as a computer. PC, or laptop. The computing device 106 can receive the spectropolarimetric data packages from the spectropolarimetric camera 102. In some embodiments, the computing device 106 can be configured to control the settings of one or more of the spectropolarimetric camera 102, including image settings as well as scanning and positioning settings.

In some embodiments, the computing device 106 can identify or receive spectropolarimetric data packages of regions of the eye 105. In some embodiments, the computing device 106 can receive spectropolarimetric data packages including a multi-dimensional spectropolarimetric measurement of the eye. In some embodiments, the computing device 106 can transform the multi-dimensional spectropolarimetric measurement into a multi-dimensional spectropolarimetric data package.

In some embodiments, for each of the regions, the computing device 106 can identify or receive spectropolarimetric data packages at multiple wavelength ranges. The computing device 106 can include spatial information about a corresponding region of the eye 105. The spatial information can comprise texture, formations, and patterns in the corresponding region. In some embodiments, the computing device 106 applies a pixel-wise analysis to the spectropolarimetric data packages.

In some embodiments, the computing device 106 can receive or identify polarization components in the spectropolarimetric data packages. In some embodiments, the computing device 106 can receive a multi-dimensional spectropolarimetric measurement of an eye. In some embodiments, the computing device 106 can transform the multi-dimensional spectropolarimetric measurement into a multi-dimensional spectropolarimetric data package. In some embodiments, the computing device 106 can identify the polarization of light in two or more orthogonal components and can be commonly represented in the form of a Mueller matrix. In some embodiments, the computing device 106 can identify polarization linear or circular. Common polarization measurements include depolarization, retardation (circular, linear, and elliptical), and diattenuation (circular and linear: also referred as dichroism). Other polarization measures included polarizance, anisotropy, and Q metric. In some embodiments, the computing device 106 can identify spectropolarimetric components that can relate to an anatomical location. In some embodiments, the spectropolarimetric data packages can include spectropolarimetric components related to certain pathologies, such as patterns, formations, or textures in the imaged region that can be seen based on the different wavelength or different polarizations at which the images are captured. In some embodiments, such pathologies may be observed or identified by the computing device 106.

In some embodiments, the computing device 106 can apply preprocessing to the spectropolarimetric data packages to extract normal image components and the polarization components from the spectropolarimetric data packages. In some embodiments, the computing device 106 can apply filtering to the spectropolarimetric data packages to extract normal image components and the polarization components from the spectropolarimetric data packages.

The computing device 106 can implement a machine learning algorithm through one or more neural networks. The machine learning algorithm can include logistic regression, variational autoencoding, convolutional neural networks, transformers, or other statistical techniques used to identify and discern neurodegenerative disease-associated pathologies. The machine learning algorithm can also use spectropolarimetric scattering models, other scattering models, or optical physics models validated a priori. The neural network may comprise a plurality of layers, some of which are defined and some of which are undefined (or hidden). In some embodiments, the neural network can be a supervised learning neural network.

In some embodiments, the neural network may include a neural network input layer, one or more neural network middle hidden layers, and a neural network output layer. Each of the neural network layers include a plurality of nodes (or neurons). The nodes of the neural network layers are connected, typically in series. The output of each node in each neural network layer is connected to the input of one or more nodes in a subsequent neural network layer.

In some embodiments, the four-dimensional array or four-dimensional data of the spectropolarimetric data packages is fed into the neural networks for analysis. In some embodiments, the computing device 106 applies one or more neural networks to each dimension of the multi-dimensional spectropolarimetric data package to generate a dimensional output for each dimension of the multi-dimensional spectropolarimetric data package. In some embodiments, the pixels or voxels from the spectropolarimetric data packages can be fed into the neural networks without scaling or filtering them down. The computing device 106 can process the spectropolarimetric data packages. In some embodiments, the computing device 106 can process all the data collectively instead of individually. In some embodiments, the computing device 106 can process each slice of the data separately without slicing the data.

In some embodiments, kernels of the convolutional neural networks maintained by the computing device 106 can analyze the spectropolarimetric data packages. In some embodiments, the computing device 106 maintains a neural network for processing four polarimetric states. In some embodiments, the computing device 106 maintains a network that has four inputs. In some embodiments, each of the four inputs receives one of the 4 components of the spectropolarimetric data package. In some embodiments, each input receives a 3-D cube of the spectrum. In some embodiments, the four inputs receive data at the same time to the network. In some embodiments, the four inputs receive data with different measurements. In some embodiments, the neural networks process each input separately.

The inputs of each node in the neural network may be scalar, vectors, matrices, objects, data structures and/or other items or references thereto. Each node may store its respective activation function, weight (if any) and bias factors (if any) independent of other nodes. In some embodiments, the decision of one or more output nodes of the neural network output layer can be calculated or determined using a scoring function and/or decision tree function, using the determined weight and bias factors.

In some embodiments, each node is a logical programming unit that performs an activation function (also known as a transfer function) for transforming or manipulating data based on its inputs, a weight (if any) and bias factor(s) (if any) to generate an output. The activation function of each node results in a particular output in response to input(s), weight(s), and bias factor(s).

In some embodiments, as the spectropolarimetric data package progresses through the neural networks, it is transformed away from the image space (e.g., what each axis represents) and into a latent space. For example, each axis can be represented as an internal record such that the four dimensions are recorded but represented differently from the first provided spectropolarimetric data packages. In some embodiments, the initial kernels match the same dimensions of the integral of the spectropolarimetric data packages.

In some embodiments, the computing device 106 sorts or filters a smaller number of features. In some embodiments, the computing device 106 extracts potentially relevant data points from the spectropolarimetric data package. In some embodiments, the computing device 106 maintains all the coordinate image data into the neural network. In some embodiments, the computing device 106 join the insights of the filtered features together to output a classification.

At step 304, the computing device 106 can use the images to classify the patient into a category conveying a status about a disease. The status can indicate whether the disease is present. For example, the computing device 106 can determine or identify one or more patterns indicative of pathology (e.g., presence or absence of biomarkers indicative of a neurological disease).

In some embodiments, the computing device 106 can fit the inputs of the four-dimensional cube such that four dimensional kernels can include several different architectures that fit to show different features as input to a classifier that can match an input image to diagnostic classes. In some embodiments, the classifier of the computing device 106 can select the output as a disease classification of the multi-dimensional spectropolarimetric data package by combining each dimensional output of each dimension of the multi-dimensional spectropolarimetric data package.

In some embodiments, the classifier of the computing device 106 can execute regression models. In some embodiments, the computing device 106 can compare spectropolarimetric components of a subject to a database of spectropolarimetric components of the same subject to see a progression (regression). The progression (regression) of the subject can also be compared to other population cohorts and their historical progression (regression). For example, a regression model can be used to identify a level of cataracts of a person.

In some embodiments, the classification (output of the neural network) can be one or more conclusions about whether the subject has a neurodegenerative pathology, or a precursor to a neurodegenerative pathology, or is pre-screened for potential of neurodegenerative pathology and requires further investigation. Such neurodegenerative pathology conclusions can be based on one or a plurality of pathologies classified by the neural network, and determined or calculated using a combined weighted score, scorecard, or probabilistic determination.

For example, the presence or probabilistic classification of both Amyloid Beta and Tau neurofibrillary tangles may lead to a higher probability conclusion of a neurodegenerative pathology.

In some embodiments, the conclusions can also be based on the changes over time of the physiology of the subject, for example by comparing with previous spectropolarimetric or spectroscopy information of the subject.

In some embodiments, the hyperspectral, polarimetric, or reflectance information is also used as input information to the neural network maintained by the computing device 106, which helps classify neurodegenerative pathologies.

In some embodiments, the computing device 106 can maintain a segmentation model. In some embodiments, the computing device 106 can use one model to the segmentation and that model can be fed into another model as a feature to another diagnostic tool. In some embodiments, the computing device 106 can perform semantic segmentation to identify different parts of the eye 105. In some embodiments, the computing device 106 can perform semantic segmentation based on the spectropolarimetric data packages. In some embodiments, the computing device 106 can perform semantic segmentation based on images of the eye 105.

In some embodiments, the computing device 106 can combine the segmentation with the spectropolarimetric data, which can be a useful approach when feeding both sets of data into the neural networks to classify different diseases. In some embodiments, the computing device 106 can apply one or more classification networks to the segmentation measurement and the multi-dimensional spectropolarimetric measurement to select a disease classification of the eye 105. For example, different diseases can be mapped using the classification networks. In some embodiments, the computing device 106 can provide or output the segmentation data, which may be apart from the spectropolarimetric data that are dividing the segmentation data, to recognize the differences between different regions of the eye 105. In some embodiments, the computing device 106 can perform the segmentation with an automated segmentation algorithm.

In some embodiments, the computing device 106 can regionally segment the eye to identify pixels in the various components of the eye 105, including the optic disc (nerve head), retina, and fovea. For example, the segmentation data can be used to identify properties of blood vessels compared to the rest of the tissue in the eye 105. In some embodiments, the computing device 106 can perform semantic segmentation to identify cataracts as well as anatomical pathology. In some embodiments, the computing device 106 can use semantic segmentation to identify or determine the existence of advanced macular degeneration (AMD). In some embodiments, the computing device 106 can use semantic segmentation to identify the age and sex of a person based on their retina. In some embodiments, the computing device 106 can use semantic segmentation to identify one or more AD-associated pathologies, including, but not limited to, protein aggregates, the protein aggregates including at least one of: Tau neurofibrillary tangles, Amyloid Beta deposits, soluble Amyloid Beta aggregates, or Amyloid precursor protein.

In some embodiments, the computing device 106 can use a first imaging modality to identify the locations of blood vessels in the eye 105 (e.g., based on spatial components in an image and/or by detecting blood flow from the image). For example, the vessels or arteries associated with diseases can be detected in the fovea region by using semantic segmentation. In some embodiments, the computing device 106 can use a second imaging modality to analyze the spectropolarimetric components of the blood vessels where the neurological disorders or pathologies may be more likely to be evident. In some embodiments, the computing device 106 can segment the regions within the optic disc to identify more specific components, including a temporal rim, nasal rim, inferior rim, superior rim, and cup regions.

In some embodiments, the computer device 106 can identify the change in polarization as a function of wavelength between the light illuminating the eye 105 and the light returning from the eye 105 and/or the optical density and/or reflectance of the acquired spectra to determine the presence or absence of the amyloid or tau formations. In some embodiments, the computer device 106 can use polarization measurements at different wavelengths from different regions of the eye 105. In some embodiments, the computer device 106 can identify wavelength range(s) of the wavelength-dependent polarization changes, as well as wavelength-dependent polarization change ratios, that have significance about the presence or absence of amyloid or tau formations. The wavelength range(s) of the optical density and reflectance, as well as optical density and reflectance ratios may have significance about the presence or absence of amyloid or tau formation.

In some embodiments, the computing device 106 can use the spectropolarimetric components to identify or characterize properties of tissue polarization and birefringence that are spectrally dependent. In some embodiments, the computing device 106 can generate or produce the spectropolarimetric data packages by combining the polarization component measurements for each wavelength at each pixel into a single intensity value for each wavelength at each pixel (or if the different polarization components are measured on different pixels, then by combining them into a single compound pixel). In some embodiments, the computing device 106 can generate or produce a purely spatial image from the spectropolarimetric data packages by combining the individual wavelength component measurements at each pixel into a single intensity value for that pixel.

In some embodiments, the computing device 106 can tag or register different spectropolarimetric data packages to ensure alignment in space between the spectropolarimetric data packages. The computing device 106 can identify corresponding spatial components in two or more images and shift (translate and/or rotate using either rigid or elastic transformations) the positions of the spectropolarimetric data packages so that those spatial components overlap in a co-registered coordinate system. The calculated shift for each spectropolarimetric image to the co-registered coordinate system can then be used to shift subsequent spectropolarimetric data packages.

At step 306, the computing device 106 provides the output of the category of indicating the status of on the disease. For example, the computing device 106 can provide a diagnosis for one or more pathologies. The computing device 106 can allow for the identification of at-risk populations, diagnosis, and tracking of subject response to treatments. In some embodiments, the computing device 106 can detect protein aggregates of Aβ, tau, phosphorylated tau, and other neuronal proteins indicative of a neurodegenerative disease, in particular Alzheimer's disease. In some embodiments, the detected protein aggregates can include at least one of Tau neurofibrillary tangles. Amyloid Beta deposits or plagues, soluble Amyloid Beta aggregates, or Amyloid precursor protein. These detected proteins can suggest a pathology in the brain as they can be correlated to brain amyloid and/or brain tau.

In some embodiments, the computing device 106 can detect the existence of one or more of AD associated pathologies or pathologies associated with neurodegenerative diseases (e.g., Parkinson's disease (PD), amyotrophic lateral sclerosis (ALS), multiple sclerosis. Prion disease. Motor neuron diseases (MND), Huntington's disease (HD), Spinocerebellar ataxia (SCA), Spinal muscular atrophy (SMA), cerebral amyloid angiopathy (CAA), other forms of dementia, and similar diseases of the brain or the nervous system). In some embodiments, the computing device 106 can detect other conditions in and related to the eye 105 such as age-related macular degeneration and glaucoma.

In some embodiments, the computing device 106 can detect biomarkers indicative of tau pathologies or tauopathies, including, without limitation, total (T-tau), Tau PET, and phosphorylated tau (P-tau). In some embodiments, the biomarkers indicative of a Tauopathy include, but are not limited to, phosphorylated paired helical filament tau (pTau), Early Tau phosphorylation. Late Tau phosphorylation, pTau 181, pTau217, pTau231, total Tau, Plasma Aß 42/40, Neurofibrillary tangles (NFTs) and aggregation of misfolded tau protein. In some embodiments, neurofilament light protein (NFL), neurofilaments (NFs) or abnormal/elevated neurofilament light protein (NFL) concentration can be detected. In some embodiments, the computing device 106 can detect surrogate markers of a neurodegenerative disorder or neuronal injury indicative of retinal and optic nerve volume loss or changes, degeneration within the neurosensory retina, or optic disc axonal injury.

In some embodiments, the computing device 106 can detect an inflammatory response or neuroinflammation that may be indicative of neurodegenerative disease. In some embodiments, the computing device 106 can detect such inflammatory response in the retinal tissue. In some embodiments, the responses include, but are not limited to, retinal microglia activation, degenerating ganglion cells (ganglion neuron degeneration) or astrocyte activation. In some embodiments, protein aggregates or biomarkers include alpha synuclein and TDP43 (TAR DNA binding protein-43) and others described, for example, in Biomarkers for tau pathology (Molecular and Cellular Neuroscience, Volume 97, June 2019, Pages 18-33), incorporated herein by reference in its entirety.

In some embodiments, the computing device 106 can detect the presence or absence of protein aggregates or biomarkers indicative of neurodegenerative diseases in the subject's tissue of the eye 105, brain tissue, tissues of the central nervous system, peripheral nervous system, or in the cerebrospinal fluid (CSF) or any other tissue where such formations or their biomarkers occur. In some embodiments, the computing device 106 detects protein aggregates or biomarkers indicative of one or more neurodegenerative diseases without using a dye or ligand.

In individuals with neurological diseases such as Alzheimer's disease, both the amyloid and tau levels in the brain are elevated before the onset of symptoms. The levels of amyloid and tau are correlated in that subjects who develop AD tend to have biomarker evidence of elevated amyloid deposition biomarkers (which is detected via abnormal amyloid PET scan or low CSF Ab42 or Ab42/Ab40) ratio) as the identifiable evidence of abnormality, followed by biomarker evidence of pathologic tau (which is detected via CSF phosphorylated tau, and Tau PET). This may be due to amyloid pathology inducing changes in soluble tau release, leading to tau aggregation later. The changes enable the predictive abilities for prediction of amyloid status and the developed model for amyloid status to be considered valid for predicting the tau status of an individual because the tau and amyloid levels are correlated.

The capabilities of spectropolarimetric imaging for detection of tau protein in the retina are important because the eye 105 is an extension of the central nervous system, linked by the optic nerve directly to the brain, proteins produced in the brain as part of neurological diseases such as Alzheimer's disease progression such as beta amyloid and tau migrate from the brain to the fundus of the eye 105. The detection of these proteins in the eye 105 can suggest the presence or absence of these proteins in the brain and corresponding risk of developing neurological diseases such as Alzheimer's disease. The ability to measure tau in brain tissue shows the feasibility to measure tau in ocular tissues, such the retina and optic disc, and use these measures as a proxy for the levels of tau in the brain.

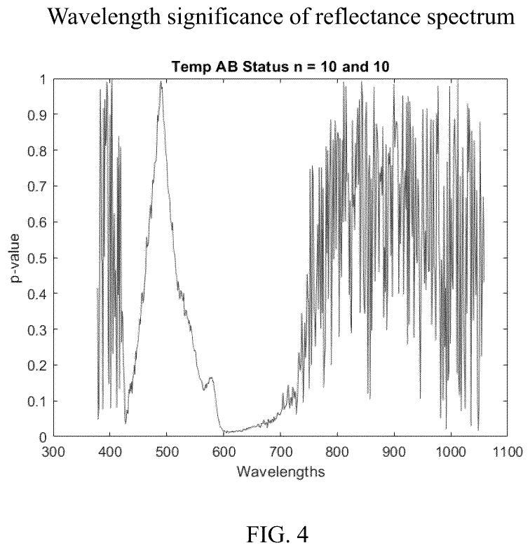

Now referring to FIG. 4, the computing device 106 can use significance plots to identify the wavelengths or wavelength ranges where the values are significant. The computing device 106 can determine a significance of the identified patterns. In some embodiments, the computing device 106 generates spatially average spectropolarimetric values (including polarization (depolarization, retardation, and diattenuation), optical density, and reflectance) in the respective regions (disc regions, retina, fovea, etc.) to produce an average spectrum for each region, Sₐᵥₑ, where Sₐᵥₑ is the mean of all the pixel values contained in the region.

The average spectrum from each region can be used along with a previously acquired white reference spectrum S_{ref} to calculate the average optical density (OD) for each spectropolarimetric layer in the spectropolarimetric data package. The white reference spectrum S_{ref} is acquired by the spectropolarimetric camera 102 imaging a diffuse broadband reflectance standard target. In some embodiments, the spectropolarimetric camera 102 images a Spectralon target and the computing device 106 calculates the mean spectrum of the acquired spectropolarimetric data package. OD is a measure of how optically absorbing a material is, a higher OD value corresponds to a higher level of absorption by the material. The optical density of each region can be calculated as:

$$OD = log ( {S}_{ref} / {S}_{ave} ) .$$

The OD spectrum is normalized. The computing device 106 can divide the spectrum by a value between 700 and 1000 nm, or by a value at some other wavelength, or through signal normalization techniques such as standard normal variate (SNV) normalization.

In some embodiments, the reflectance spectrum R can be calculated. The reflectance is a measure of the optical reflectance of the material being imaged, a higher value for reflectance shows the material has higher optical reflecting properties. In some embodiments, the reflectance R can be calculated by:

$$R = log ( {S}_{ave} / {S}_{ref} ) .$$

In some embodiments, the reflectance spectrum is normalized to a wavelength between 700 nm and 850 nm. Or reflectance spectrum can be normalized via standard normal variate (SNV), minimum maximum, or other normalization techniques.

To assess which wavelengths or wavelength-ranges the polarization, OD, and R values correlate significantly with amyloid and tau status of a subject's status, a statistical significance test (e.g., students t-test) can be performed for the polarization, R, and OD values at each wavelength for spectra acquired from a group of subjects having negative and positive amyloid and tau status. The statistical significance test identifies if the values at each wavelength are significantly correlated with the amyloid and/or tau status of the subjects. Examples of significance tests include t-tests, Pearson correlation, Spearman correlation, Chi-Square, ANOVA, among many others.

In some embodiments, the computing device 106 uses pixels in the temporal region of the optic disc in the calculations to determine a metric indicative of amyloid and tau status of the individual pixels. In some embodiments, the values extracted from other regions can contain information related to the amyloid or tau status of the individual as well as information related to other ocular or systemic pathologies. In some embodiments, the extracted values from other regions may contain amyloid or tau protein deposits measurable through spectropolarimetric and/or polarization imaging or could exhibit the effects of these proteins on the tissues. In some embodiments, other regions may contain information related to other pathologies of the fundus, such as macular degeneration, glaucoma, and diabetic retinopathy. In some embodiments, this information is measurable through spectropolarimetric and/or polarization imaging.

In some embodiments, the significance is defined as having a p-value lower than 0.05, which is evident in wavelengths ranges 600-700 nm for the OD spectra. Once the wavelengths or wavelength ranges of significance have been determined, the computing device 106 can be optimized (for cost, size, speed, and other factors) by causing the spectropolarimetric camera 102, light sources 103, and/or polarizer 120 to only measure light at those wavelengths or wavelength ranges. The computing device 106 can make the selections based on machine learning and/or artificial intelligence techniques in optimizing the sensor design.

The computing device 106 can use the significance plots to evaluate the significance of spectropolarimetric values (including polarization, optical density, and reflectance) for any status of the sample being measured and is not restricted to amyloid or tau status of a human.

In some embodiments, the computing device 106 can use the significance plots to identify ocular pathologies (e.g., macular degeneration, diabetic retinopathy, and glaucoma) and extract measurements of tissues (e.g., skin, muscle, tendon, blood vessels, and other tissues).

In some embodiments, the computing device 106 can identify and analyze tissues of other organisms. The approach would be the same for these different pathologies and/or tissue types but the spectropolarimetric values determined to be significant may be different in each case. In some cases, it may be desirable to analyze samples for more than one pathology or disease state to identify and diagnose subjects with more than one condition, or to identify subjects with a first disease state and exclude them from analysis of a second disease state if it is known that the presence of the first disease state would affect the results of the analysis for the second disease state.

In some embodiments, the computing device 106 identifies or assesses a significance of polarization. R and OD values, a significance of ratios of polarization, and R and OD values at various wavelengths. The computing device 106 can divide each wavelength dependent spectropolarimetric value of polarization. R and OD, by all other polarization. R and OD values to assess all ratios for statistical significance. The results of these significance ratios can be plotted as a 2D image with the numerator and denominator of the wavelengths as the X and Y axis.

Now referring to FIG. 5, shown is a method for assessing significance of values at each wavelength and the ratios of values at each wavelength. This process can be generalized to any spectropolarimetric type of data to assess the signal for significance with a parameter of interest (in this case the amyloid and/or tau status of an individual). As shown in the flowchart in FIG. 5, a method for wavelength feature identification for values at each wavelength and for ratios of values at each wavelength can include the steps of measuring a spectropolarimetric signal (step 502) and correcting and/or calibrating the spectropolarimetric signal (step 504). The significance of spectropolarimetric values at each wavelength to a parameter is calculated (step 506), and if that calculated value is found to be significant, that associated wavelength is noted as significant (step 508). If it is not found to be significant, a ratio of each wavelength to all the other wavelengths can be calculated (step 512), and the significance of spectropolarimetric values at each wavelength ratio to a parameter is calculated (step 514). If that calculation is found to be significant (step 516), the wavelength ratio can be noted as significant. Once a wavelength or wavelength ratio is found to be significant (step 518), a scan or image associated with the individual can be tested by comparing to a control image at the significant wavelength or wavelength ratio (step 520)). In some embodiments, the significant wavelengths are the same for the entire eye 105 and in other embodiments the significant wavelengths are different for different regions of the eye 105.

In some embodiments, the computing device 106 identifies the significance of the ratios of spectropolarimetric values for any status of the sample being measured and is not restricted to amyloid or tau status of a human. In some embodiments, the computing device 106 can identify other ocular pathologies. In some embodiments, the computing device 106 can identify pathologies in tissues of other organisms. The computing device 106 can analyze the spectropolarimetric data packages for these different pathologies and/or tissue types but the ratios of spectropolarimetric values determined to be significant may be different in each case.

In some embodiments, the spectropolarimetric camera 102 may acquire or generate spectropolarimetric measurements in the mid-IR wavelength range, specifically in the range of 5900 nm-6207 nm and/or 6038 nm-6135 nm with specific interest at 6053 nm and 6105 nm wavelengths. The amyloid-β aggregation process can span many years and during this process the amyloid-β presents in both soluble and plaque form, folded into α-helix and β-sheet structures, with the relative concentrations of those structures changing over time. Those structures and their concentration ratio are a function of the progression of the aggregation process, which is an important biomarker to make clinical assessments of AD presence and progression. The different folding structures of this protein are known to have different spectral-dependent polarization changes as well as different spectropolarimetric absorbance and reflectance, with the amyloid's a-helix structure having a peak at 6053 nm while the β-sheet of that protein has a peak at 6105 nm. The peak absorbance and reflectance observed in the eye 105 can be an indication of the concentration ratio. For example, a peak absorbance around 6079 nm would be evidence of a balanced mixture. The more that the ratio tends towards the peak absorbance of one of the structures, the greater the concentration of the structure having that peak absorbance. In some embodiments, the spectropolarimetric camera 102 is a spectra imager identifying the range of 6038 nm-6135 nm to measure these biomarkers. Other important wavelengths are 5900 nm. 6060 nm. 6150 nm, and 6207 nm which are related to structures that have clinical importance as well (such as β-hairpin, β-sheets, amyloid β1-42 fibrils. Tyr, and Phe amino acid).

The computing device 106 can use feature identification to identify, in the spectropolarimetric data packages, wavelength ranges of spectropolarimetric components and wavelength ranges of spectropolarimetric value ratios for the analysis of any sample of interest and is not limited to spectroscopy and/or polarimetry of the biological tissue. In some embodiments, the computing device 106 can be used for pharmaceutical process monitoring, industrial process monitoring, hazardous material identification, explosive material identification, and food process monitoring. In some embodiments, the computing device 106 can identify optical and spectroscopic modalities for the exploration of significant features with a property of interest in the sample. In some embodiments, the computing device 106 can identify or utilize various optical modalities, including Raman spectroscopy, fluorescence spectroscopy, laser-induced breakdown spectroscopy. Fourier transform infrared (FTIR) spectroscopy, nonlinear optical microscopy (e.g., multiphoton microscopy, harmonic generation microscopy), and other optical modalities.

The significant wavelength regions of the polarization. R, and OD spectra and the ratios of polarization. R, and OD spectra can be identified as significant features of the spectra and used as inputs to a machine learning (ML) or artificial intelligence (AI) algorithm to predict amyloid or tau status of an individual based on a model trained on spectropolarimetric data packages acquired from individuals with a known status. The ML models can include logistic regression, decision tree, random forest, linear discriminant analysis, neural networks (including convolutional neural networks and transformers), naïve bayes classifier, nearest neighbor classifier or other ML or AI techniques.

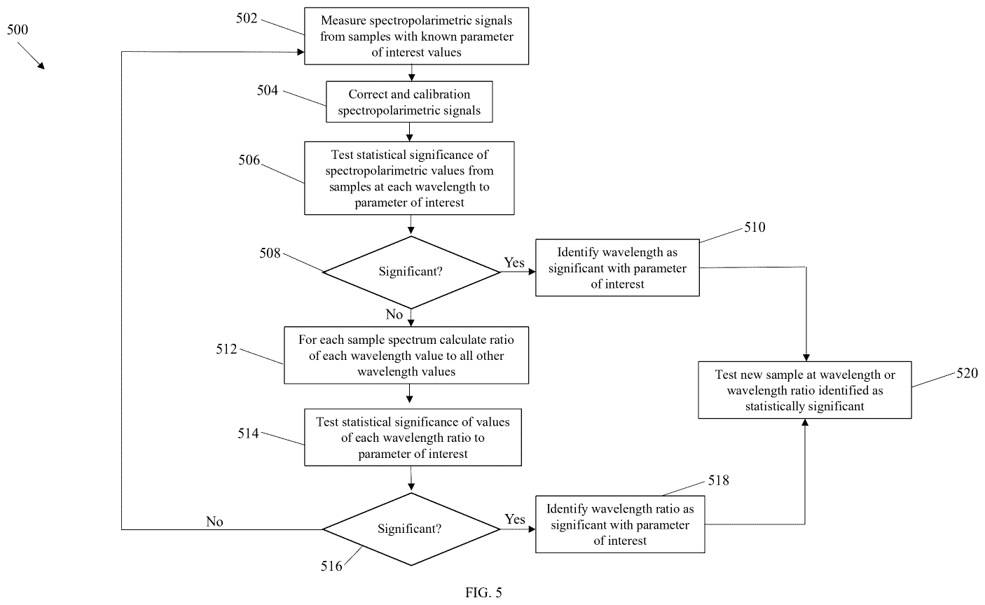

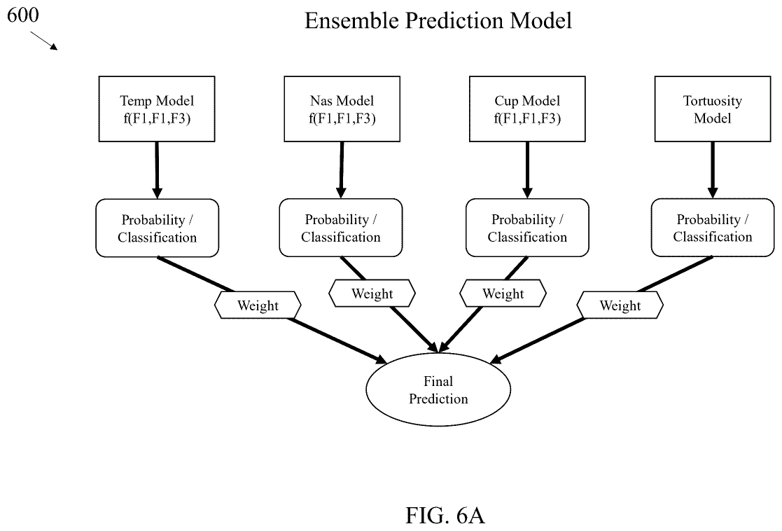

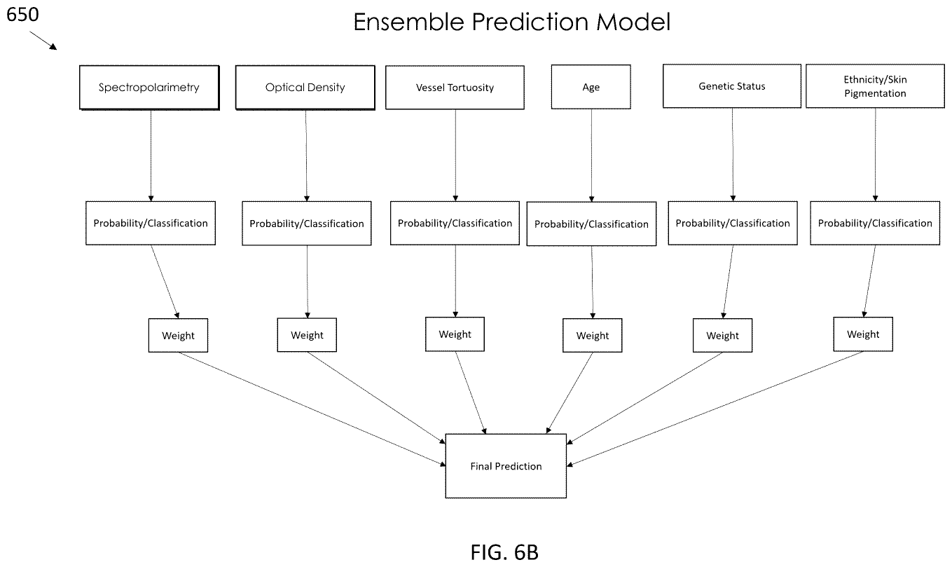

Now referring to FIG. 6A and FIG. 6B, the computing device 106 can utilize an ensemble technique. The features identified by statistical significance testing previously described along with other features extracted from spectropolarimetric imaging (such as blood vessel tortuosity) are used as features to train individual ML models, and the outputs of the models are considered in combination with various weights being applied to outputs of the models to calculate a combined output to predict the status of the individual.

The significant wavelength regions of the polarization. R, and OD spectra and the ratios of polarization. R, and OD spectra are identified as significant features of the spectropolarimetric data packages and spectra and used as inputs to a machine learning (ML) or artificial intelligence (AI) algorithm to predict amyloid or tau status of an individual based on a model trained on spectropolarimetric data packages and spectra acquired from individuals with a known status. The ML models can include logistic regression, decision tree, random forest, linear discriminant analysis, neural networks (including convolutional neural networks and transformers), naïve bayes classifier, nearest neighbor classifier or other ML or AI techniques. The computing device 106 can identify the features by statistically significant testing previously described, along with other features extracted from spectropolarimetric imaging (such as blood vessel tortuosity) are used to train individual ML models, the outputs of these models are considered in combination with various weights being applied to outputs of the models to calculate a combined output to predict the status of the individual.

Such an ensemble model can include features extracted from spectropolarimetric data packages including but not limited to tissue/vessel oxygenation, vessel tortuosity, cup-to-disc ratio, retinal nerve fiber layer thickness, and image texture metrics. In some embodiments, demographic and other medical information on the individual could be used as an input to such an ensemble model, including but not limited to age, sex, ocular pathologies, comorbidities, lens status (natural vs artificial), previous ocular surgeries, if dilation drops used during imaging, and any other demographics or health information.

In an ensemble or ‘multimodal’ model, the different data types (e.g., spectropolarimetric, polarimetric, spectral, spatial, demographic, etc.) could each be processed by different machine learning or artificial intelligence algorithms independently, with the outputs of those algorithms used as inputs to algorithms, or multiple data types (e.g., combined spectropolarimetric components) could be used by the same algorithm directly to produce an output based on an evaluation in multiple data domains. In some cases, this is advantageous for capturing correlations in data between multiple domains, and these correlations can be lost if the analysis to combine data types is performed only using the extracted outputs from independent algorithms rather than the full data sets.

In an ensemble model using spectropolarimetric data packages, the analysis algorithms may use the entire spectropolarimetric data package, portions of the spectropolarimetric data package, or only segmented sections of the spectropolarimetric data package. This type of ensemble model can be used even if features such as the optic disc, which is often used as a reference for performing segmentation, are not present in the spectropolarimetric data package. This type of model may analyze the entire spectropolarimetric data package, such as when the relevant information is unlocalized and appears over large areas of the image, or it may only analyze portions of the image or segmented sections of the spectropolarimetric data package, such as when the relevant information is highly localized.

In some embodiments, the computing device 106 can use machine learning or artificial intelligence algorithms to make pixel-wise predictions (for 2 spatial dimensions) or voxel-wise (for 3 spatial dimensions) analysis of each spectropolarimetric data package. Pixel-wise predictions are based on the spectropolarimetric data (including polarization, optical density, and reflectance) for each pixel and the relationship between the spectropolarimetric data from two or more adjacent or nearby pixels, rather than only data from a single pixel or the overall spectropolarimetric data of a group of pixels together, as is the case in channel-wise prediction. Pixel-wise predictions can be used to avoid having the algorithm rely on multiple pieces of information that are spatially disconnected from each other, which is important when the relevant information is regional since a pixel-wise prediction reduces overfitting of the model and improves performance. Pixel-wise prediction can enable a better ability to test, validate, and explain the algorithm outputs instead of solely relying on attention maps or input distortion, which allows for verification that the location of the signal in the spectropolarimetric data package corresponds to the correct area where the signal is expected to be, thus opening the “black-box” of the algorithm, and making the output predictions more transparent.

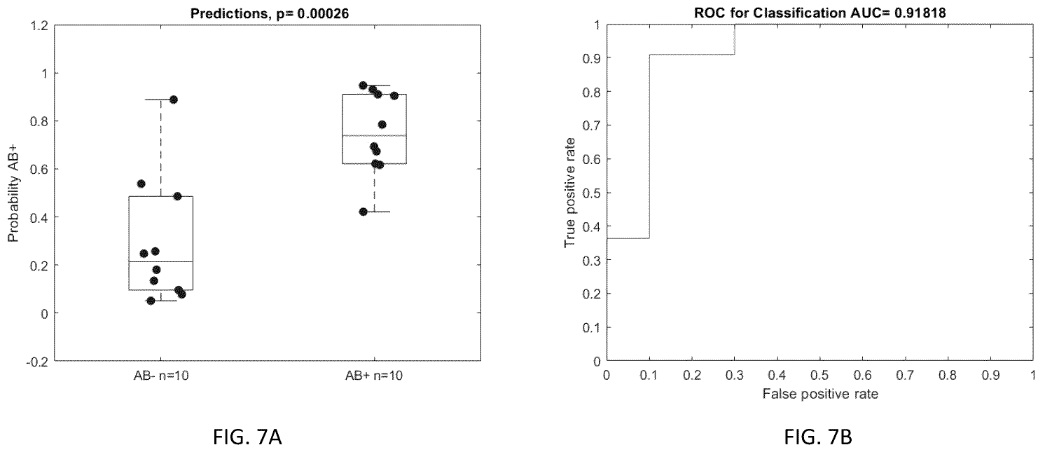

FIG. 7A shows the predictions of an individual's amyloid status based on an ensemble model using features from the R and OD ratio features from their eye 105 for ten individuals with negative amyloid status and 10 individuals with positive amyloid status. As shown in FIG. 7B, the computing device 106 evaluated the results of this model through a receiver operator curve (ROC) providing an area under the curve (AUC) of greater than 0.9, indicating high predictive capabilities of the developed model for amyloid status.

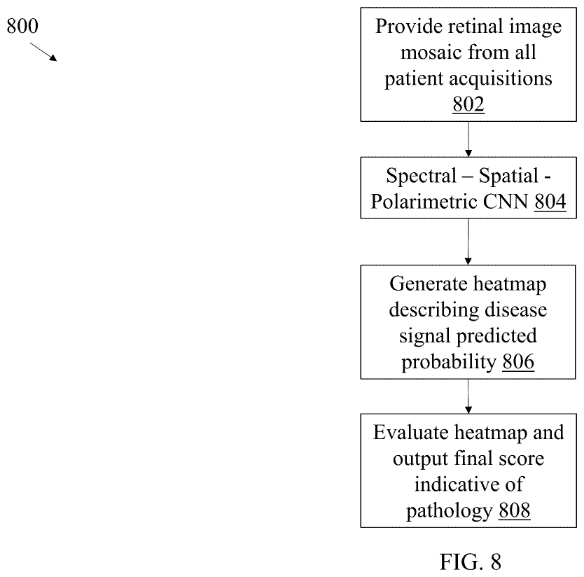

FIG. 8 illustrates a method for processing spectropolarimetric data packages. The computing device 106 can receive and analyze spectropolarimetric data packages generated by the spectropolarimetric camera 102. A retinal image mosaic is provided from the spectropolarimetric data packages acquired from a subject (step 802). In some embodiments, the computing device 106 can be configured to obtain, request, or receive a retinal image mosaic comprising the spectropolarimetric data packages of the eye 105.

The computing device 106 can use a spectropolarimetric convolutional neural network (CNN) (step 804) to generate a heatmap. In some embodiments, the computing device 106 can generate a digital representation indicative of a presence or absence of the biomarkers in the one or more regions of the eye 105. In some embodiments, the algorithm can be configured with a CNN to accept, receive, or analyze the spectropolarimetric data packages. In some embodiments, the algorithm can be configured with the layers to support the spectropolarimetric analysis. In some embodiments, the algorithm can change the width, depth, and length of the networks according to the capacity needed for detecting signal in the spectropolarimetric data packages. In some embodiments, the algorithm can be configured by replacing the last layers (near the output) so that the last feature tensor of the network is pooled in a pixel-wise instead of channel-wise.

The inputs to the neural network are the spectropolarimetric data packages captured by the spectropolarimetric camera 102 from both eyes for each subject. For example, for each eye 105, there can be 7 collections of images centered at different anatomical locations on the eye 105. The locations can include one or more of the optic disks, the center of the retina, the fovea, superior, inferior, temporal, and nasal. In some embodiments, for each location line, the spectropolarimetric camera 102 can acquire or generate spectropolarimetric components that crosses at the center of the image in a horizontal line. In some embodiments, for each eye 105, a color Fundus image can be taken or captured by the spectropolarimetric camera 102 to be used in AI to gain more insight and for the ophthalmologist to determine diagnosis and pathologies such as retinopathy, macular degeneration, glaucoma, cataract, hypertension, etc.

In some embodiments, the algorithm can be trained using a database of corresponding data from subjects with known disease state. A plurality of subjects and a control set of health individuals can be used. Data can be acquired from each subject, and the data and/or images collected can be pre-processed. Low quality images can be excluded and the spectropolarimetric data packages can be normalized. The data can be split into three sets for training, validation, and testing, using multiple folds in a cross-validation methodology and ensemble different models from different folds together using the training data before testing on the test set. In some embodiments, the training set is exposed to AI during training and is used for the actual learning, the testing set is frequently used during the train process to evaluate the performance of the model on unseen data, and the validation set can be held out, separate to the developers of the AI, and is only used one time to validate the model on new data.

In some embodiments, the training can be difficult on a per-image level because the relevant information isn't apparent or doesn't exist in all the spectropolarimetric data packages acquired from a single subject. For example, training is difficult if the information is apparent in an image of the optic-disc but not a spectropolarimetric data package for the fovea, or if it is apparent in the left eye but not in the right eye. Training can become difficult on a per image level as some labels (positive vs, negative) can be misleading to the AI. To address this problem, in some embodiments, some or all the images of a single subject are concatenated into one mosaic of images and analyzed as one whole sample. In that way, even if the signal is apparent in only one of the images, the training labels assigned to this mosaic would be correct and not mislead the AI. In some embodiments, the algorithm may be an ensemble of multiple algorithms. The spectropolarimetric camera 102 can generate or capture spectropolarimetric from a subject, poor quality images are excluded and recaptured, and the spectropolarimetric data packages are compiled into the same mosaic form as in the training data set before being used by the algorithm to produce a final score. In some embodiments, when using the AI for prediction, some subjects can be excluded based on certain clinical criteria. In some embodiments, if a certain pathology such as glaucoma or certain ethnicity were underrepresented in the training and validation sets, there can be less certainty that the AI will perform on them as required. The AI can be run on all the images and clinical data can be acquired from the subject.

The computing device 106 can identify or describe disease signal predicted probability (step 806). In some embodiments, the computing device 106 can analyze the one or more spectropolarimetric data packages to identify biomarkers indicative of a neurodegenerative pathology. In some embodiments, the spectropolarimetric data package can include spectropolarimetric components that can relate to an anatomical location. In some embodiments, the spectropolarimetric data package can include spectropolarimetric components related to patterns, formations, or textures in the imaged region that can be seen based on the different wavelength or different polarizations at which the spectropolarimetric data packages are generated. In some embodiments, certain pathology formations may be observed or identified by the computing device 106.

The different machine learning algorithms can be used to generate predictions for the various sources of data. Once all these models are trained, a prediction can be generated from an ensemble model, which combines all the models in which data is available for the given individual. The final prediction can be a weighted combination of the outputs from the available models. The weight given to each model can be based on how significantly the prediction from that model correlates with the amyloid or tau status of the subjects in the training set. These weights can be adjusted as more data becomes available. The ensemble model can advantageously deal with the reality everyone will have a different combination of data available to make the prediction (e.g., some subjects may have data from the temporal rim but not the inferior rim or vice versa).

In some embodiments, the computing device 106 can calculate a saturation ratio, defined as the number of saturated data points (e.g., at the maximum value of the measurement device) divided by the number of data points of an input image. The computing device 106 can reject, identify, or tag spectropolarimetric data packages with a saturation above a preset threshold since too much saturation results in a loss of information. A data point could be the overall intensity value measured at a pixel of the image, or it could be the intensity of only one or more spectropolarimetric (wavelength) or spectropolarimetric components at that pixel.

In some embodiments, not all saturation degrades the prediction power of the algorithms, and some saturation below the preset threshold can even, in some embodiments, improve it to some degree. Since saturation in a few data points shows that the signal being measured is reaching the maximum range of the spectropolarimetric camera 102, which shows that the subject being imaged is illuminated and strongly reflecting such that the non-saturated data points are likely generating signals with good intensity and dynamic range that can provide a clear image and an accurate result. If the data points are not saturated at a level satisfying a predetermined threshold, the subject may be under-illuminated, and the full dynamic range of the spectropolarimetric camera 102 might not be utilized.

The generated heatmap can be evaluated and a final score is output that can be indicative of pathology in (step 808). For a given subject, various sources of information may be available to assist with the prediction of amyloid or tau status. For example, subjects may have spectropolarimetric and/or spectropolarimetric data from a subset of the regions, including the temporal, nasal, inferior, and superior rim of the optic disc, the cup, the fovea, along with various other spatial regions within the eye 105. Individual models can be developed using data from each of these regions. Additional models can be developed using data from the tortuosity of vessels (as determined from the color or hyperspectral or spectropolarimetric data packages), from nerve fiber thickness (as determined from OCT), from blood oxygenation, pupil dilation (or pupillary light reflex), inflammatory response, demographic data, etc. Each individual model will output the probability that a given subject has a positive or negative amyloid or tau status.

Prior to using any of the collected or calculated spectropolarimetric components, the computing device 106 can apply quality assurance criteria to ensure that the data is of sufficient quality to produce a reliable prediction output from the machine learning or artificial intelligence algorithm. In some embodiments, the computing device 106 can calculate the spectropolarimetric dynamic range, which is defined as the difference between the highest spectropolarimetric band and the lowest spectropolarimetric band in a specific pixel, for each pixel in the spectropolarimetric or hyperspectral or multispectral or spectrometer data. The computing device 106 can reject pixels with a spectropolarimetric dynamic range below a preset percentile threshold, since data with low spectropolarimetric dynamic range contains less information and may not be usable.

In some embodiments, the computing device 106 can calculate a quality assurance criterion, the blurriness, or sharpness of a spectropolarimetric data package based on changes in intensity between adjacent image pixels, and images or portions of images with blurriness above a preset threshold can be rejected, or the homogeneity of an image can be calculated based on changes in intensity across all image pixels, and images or portions of images with too much or too little homogeneity can be rejected. For each of the quality assurance criteria, the computing device can maintain or generate threshold values at which data would be accepted or rejected. While the spectropolarimetric dynamic range percentile can vary, in some embodiments the range is 20% spectropolarimetric dynamic range for the 5^{th} percentile of pixels. In some embodiments, up to 5% saturated pixels can be allowed. In some embodiments, the computing device 106 can measure blurriness with a score from 0 to 1, with a 1 being completely blurry. In some embodiments, up to 0.2 blurriness measurement can be allowed. Homogeneity can be measured by looking at histograms in sub-sections of the spectropolarimetric data package and calculating the entropy over the different histograms. In some embodiments, the threshold is set to reject spectropolarimetric data packages with entropy over 1.3. Based on the quality assurance criteria, the settings of the light source 103 and/or spectropolarimetric camera 102 can be adjusted by the computing device 106. In some embodiments, the computing device 106 can increase or decrease the intensity of the light source 103 to improve the saturation ratio, and the procedure can be repeated.

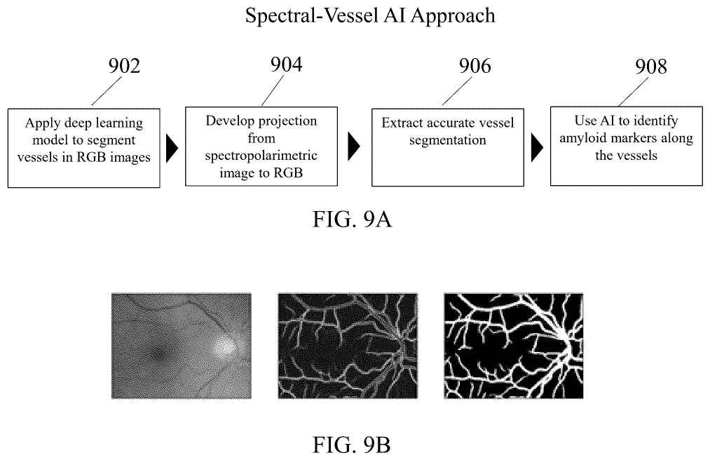

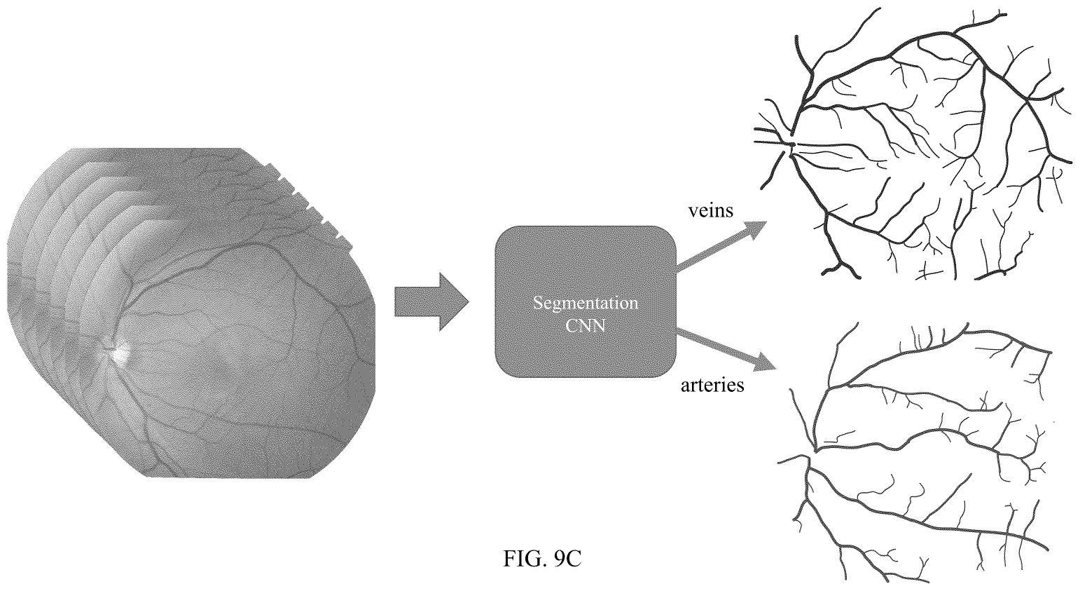

Referring now to FIG. 9A and FIG. 9B, in some embodiments, the computing device 106 can be used to analyze the vessels and vessel walls of the eye 105. An algorithm can be used to extract an accurate vessel segmentation from the captured spectropolarimetric data packages, and the AI can be used to pinpoint markers for amyloid along the vessels (e.g., vascular amyloidosis). It extends to lots of vascular changes, and other vascular pathologies that can be relevant to ocular diseases and amyloid related diseases, such as cerebral amyloid angiopathy (CAA). In some embodiments, a model can be used to segment the vessels (step 902), and a projection can be developed (step 904). Vessel segmentation can be extracted (step 906), and the AI can be used to identify amyloid markers along the vessels (step 908).

A CNN segmentation AI is developed to automatically extract vessels from the spectropolarimetric data packages. The spectropolarimetric data packages are input into the AI of the computing device 106 and the computing device 106 outputs or generates two probability maps shows the segmentation of the vessels, as shown in FIG. 9C. One map for arteries and the other for veins. Those maps correspond to spectropolarimetric data packages where the brightness of each pixel is higher where the AI predicts the existence of a vessel. These maps are binarized by a threshold, usually equal to 0.5 but can be configured. The output binary segmentations of arteries and vessels are fed to another AI that processes the structure of the vessels as well as the spectropolarimetric signature (including polarization, optical density, and/or reflectance) along the vessels to detect the biomarkers related to the disease.

It should be appreciated that the above-described processing techniques for processing the spectropolarimetric data packages are non-limiting examples. In some embodiments, the processing can be achieved by adapting deep learning algorithms to the specific dimensionality of the task for finding status of neurodegenerative disease and other indications in the spectropolarimetric retinal data. For example, convolutional neural networks generalized to 4D and adjusted kernel sizes, receptive fields, network depth, width and architecture to the size and resolution of each dimension of the spectropolarimetric data. Examples of networks that can be modified to fit the data include UNet, ResNet, ResNext, EfficientNet, DenseNet, InceptionNet, and MobileNet. A convolutional neural network adapted to spectropolarimetric data would have 4D kernels, or Tesseracts. In some embodiments. Vision Transformers (ViTs) can be adapted to spectropolarimetric data. These include ViT (Vision Transformer), DeiT (Data-efficient Image Transformers), Swin Transformer, ConVIT, TNT (Transformer in Transformer), and CoaT (Co-Scale Conv-Attentional Image Transformers).

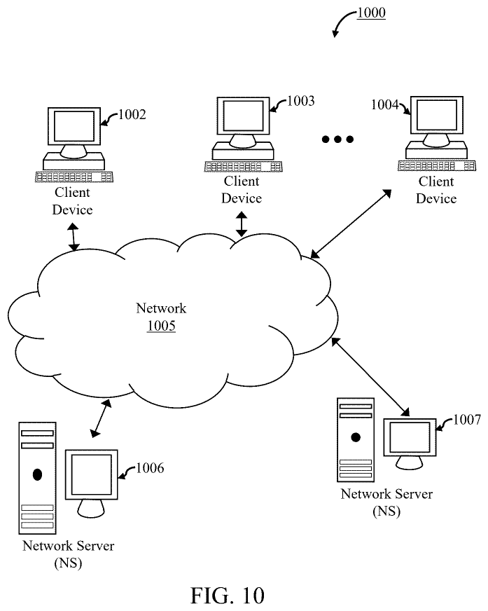

FIG. 10 depicts a block diagram of a computer-based system and platform 1000 in accordance with one or more embodiments of the present disclosure. Yet not all these components may have to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 1000 may be configured to manage many members and concurrent transactions, as detailed herein. In some embodiments, the exemplary computer-based system and platform 1000 may be based on a scalable computer and network architecture that incorporates various strategies for assessing the data, caching, searching, and/or database connection pooling. An example of the scalable architecture is an architecture that can operate multiple servers.

In some embodiments, referring to FIG. 10, member computing device 1002, member computing device 1003 through member computing device 1004 (e.g., clients) of the exemplary computer-based system and platform 1000 may include virtually any computing device able to receive and send a message over a network (e.g., cloud network), such as network 1005, to and from another computing device, such as servers 1006 and 1007, each other, and the like. In some embodiments, the member devices 1002-1004 may be personal computers, multiprocessor systems, microprocessor-based or programmable consumer electronics, network PCs, and the like. In some embodiments, one or more member devices within member devices 1002-1004 may include computing devices that typically connect using a wireless communications medium such as cell phones, smart phones, pagers, walkie talkies, radio frequency (RF) devices, infrared (IR) devices. CBs, integrated devices combining one or more of the preceding devices, or virtually any mobile computing device, and the like. In some embodiments, one or more member devices within member devices 1002-1004 may be devices that are capable of connecting using a wired or wireless communication medium such as a PDA. POCKET PC, wearable computer, a laptop, tablet, desktop computer, a netbook, a video game device, a pager, a smartphone, an ultra-mobile personal computer (UMPC), and/or any other device that is equipped to communicate over a wired and/or wireless communication medium (e.g., NFC, RFID, NBIOT, 3G, 4G, 5G, GSM, GPRS, Wi-Fi, WiMAX, CDMA, satellite, Bluetooth, ZigBee, etc.). In some embodiments, one or more member devices within member devices 1002-1004 may include may run one or more applications, such as Internet browsers, mobile applications, voice calls, video games, videoconferencing, and email, among others. In some embodiments, one or more member devices within member devices 1002-1004 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as Hypertext Markup Language (HTML), a wireless application protocol (WAP), a Handheld Device Markup Language (HDML), such as Wireless Markup Language (WML), WMLScript, XML, JavaScript, and the like. In some embodiments, a member device within member devices 1002-1004 may be specifically programmed by either Java, .Net, QT, C, C++ and/or other suitable programming language. In some embodiments, one or more member devices within member devices 1002-1004 may be specifically programmed include or execute an application to perform a variety of possible tasks, such as, without limitation, messaging functionality, browsing, searching, playing, streaming, or displaying various forms of content, including locally stored or uploaded messages, images and/or video, and/or games.

In some embodiments, the exemplary network 1005 may provide network access, data transport and/or other services to any computing device coupled to it. In some embodiments, the exemplary network 1005 may include and implement at least one specialized network architecture that may be based at least in part on one or more standards set by, for example, without limitation. Global System for Mobile communication (GSM) Association, the Internet Engineering Task Force (IETF), and the Worldwide Interoperability for Microwave Access (WiMAX) forum. In some embodiments, the exemplary network 1005 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 1005 may include and implement, as an alternative or in conjunction with one or more of the above, a WiMAX architecture defined by the WiMAX forum. In some embodiments and, optionally, in combination of any embodiment described above or below, the exemplary network 1005 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 1005 may be transmitted based at least in part on one of more communication modes such as but not limited to: NFC. RFID, Narrow Band Internet of Things (NBIOT), ZigBee, 3G, 4G, 5G, GSM, GPRS, Wi-Fi, WiMAX, CDMA, satellite and any combination thereof. In some embodiments, the exemplary network 1005 may also include mass storage, such as network attached storage (NAS), a storage area network (SAN), a content delivery network (CDN) or other forms of computer or machine-readable media.

In some embodiments, the exemplary server 1006 or the exemplary server 1007 may be a web server (or a series of servers) running a network operating system, examples of which may include but are not limited to Microsoft Windows Server. Novell NetWare, or Linux. In some embodiments, the exemplary server 1006 or the exemplary server 1007 may be used for and/or provide cloud and/or network computing. Although not shown in FIG. 18, in some embodiments, the exemplary server 1006 or the exemplary server 1007 may have connections to external systems like email. SMS messaging, text messaging, ad content providers, etc. Any of the features of the exemplary server 1006 may be also implemented in the exemplary server 1007 and vice versa.

In some embodiments, one or more of the exemplary servers 1006 and 1007 may be specifically programmed to perform, in non-limiting example, as authentication servers, search servers, email servers, social networking services servers. SMS servers. IM servers. MMS servers, exchange servers, photo-sharing services servers, advertisement providing servers, financial/banking-related services servers, travel services servers, or any similarly suitable service-base servers for users of the member computing devices 801-1004.

In some embodiments and, optionally, in combination of any embodiment described above or below, for example, one or more exemplary computing member devices 1002-1004, the exemplary server 1006, and/or the exemplary server 1007 may include a specifically programmed software module that may be configured to send, process, and receive information using a scripting language, a remote procedure call, an email, a tweet. Short Message Service (SMS), Multimedia Message Service (MMS), instant messaging (IM), internet relay chat (IRC), mIRC, Jabber, an application programming interface. Simple Object Access Protocol (SOAP) methods. Common Object Request Broker Architecture (CORBA), HTTP (Hypertext Transfer Protocol), REST (Representational State Transfer), or any combination thereof.

Non-limiting embodiments of the present disclosure are set out in the following clauses:

Clause 1. A method comprising: analyzing data from an imaging of an eye of a patient with at least one processor, the data from the imaging comprising a plurality of pixels and comprising, for each pixel, spatial, spectral, and polarimetric data generated from the imaging of the eye of the patient and wherein analyzing the data comprises analyzing the spatial, spectral, and polarimetric data for the plurality of pixels: based on the analyzing, with the at least one processor, classifying the patient into at least one category of a plurality of categories, each category of the plurality of categories indicating a status with respect to a neurodegenerative disease that affects a central nervous system; and generating, with the at least one processor, an output of one or more of the at least one category indicating the status of the patient with respect to the neurodegenerative disease that affects a central nervous system.

Clause 2. The method of clause 1, further comprising generating the spatial, spectral, and polarimetric data synchronously as a single data package of spectropolarimetric data.

Clause 3. The method of clause 1 or clause 2, wherein: the data from the imaging comprises a multi-dimensional spectropolarimetric data package; and classifying the patient into the at least one category comprises: applying, by the at least one processor, one or more neural networks to each dimension of the multi-dimensional spectropolarimetric data package to generate a dimensional output for each dimension of the multi-dimensional spectropolarimetric data package; and generating, by the at least one processor, the output as a disease classification of the multi-dimensional spectropolarimetric data package by combining each dimensional output of each dimension of the multi-dimensional spectropolarimetric data package.

Clause 4. The method of any one of clauses 1-3, wherein analyzing the data from the imaging of the eye comprises: receiving, by the at least one processor, a segmentation measurement of one or more regions of the eye; and receiving, by the at least one processor, from a spectropolarimetric camera, a multi-dimensional spectropolarimetric measurement of the eye.

Clause 5. The method of any one of clauses 1-4, wherein classifying the patient into the at least one category comprises: applying, by the at least one processor, one or more classification networks to the segmentation measurement and the multi-dimensional spectropolarimetric measurement to select a disease classification of the eye.

Clause 6. The method of any one of clauses 1-5, wherein the data from the imaging of the eye comprises spectropolarimetric data packages comprising spectropolarimetric components relating to an anatomical location of the eye.

Clause 7. The method of any one of clauses 1-6, wherein the status of the patient with respect to the neurodegenerative disease comprises a risk of the patient having or experiencing symptoms related to the neurodegenerative disease.

Clause 8. The method of any one of clauses 1-7, wherein the status of the patient with respect to the neurodegenerative disease comprises a diagnosis of the patient as having the neurodegenerative disease.

Clause 9. The method of any one of clauses 1-8, wherein the status of the patient with respect to the neurodegenerative disease comprises a progression of the neurodegenerative disease in the patient.

Clause 10. The method of any one of clauses 1-9, wherein the status of the patient with respect to the neurodegenerative disease comprises a response of the patient to preventative interventions or treatment interventions.

Clause 11. The method of any one of clauses 1-10, wherein analyzing the data comprises analyzing the spatial, spectral, and polarimetric data for the plurality of pixels with a regression model.

Clause 12. The method of any one of clauses 1-11, wherein classifying the patient into the at least one category comprises classifying the patient based on a plurality of pathologies of the neurodegenerative disease.

Clause 13. The method of any one of clauses 1-12, wherein classifying the patient based on the plurality of pathologies comprises classifying the patient based on a combined weighted score, scorecard, or probabilistic determination of each of the plurality of pathologies.

Clause 14. The method of any one of clauses 1-13, wherein analyzing the data from the imaging of the eye comprises: performing semantic segmentation to identify different parts of the eye; and combining the semantic segmentation with the spatial, spectral, and polarimetric data for the plurality of pixels.

Clause 15. The method of any one of clauses 1-14, wherein analyzing the data further comprises analyzing a relationship between the spatial, spectral, and polarimetric data for a first pixel with the spatial, spectral, and polarimetric data of two more adjacent pixels.

Clause 16. The method of any one of clauses 1-15, wherein analyzing the data comprises analyzing the spatial, spectral, and polarimetric data for the plurality of pixels with an ensemble prediction model.

Clause 17. The method of any one of clauses 1-16, wherein each individual model of the ensemble prediction model is assigned a weight based on how significantly a prediction from each individual model correlates with amyloid or tau status of the patient.

Clause 18. The method of any one of clauses 1-17, wherein analyzing the data comprises analyzing the spatial, spectral, and polarimetric data for the plurality of pixels with a convolutional neural network to generate a heatmap.

Clause 19. The method of any one of clauses 1-18, wherein analyzing the data comprises calculating a quality assurance criterion for each of the plurality of pixels.

Clause 20. The method of any one of clauses 1-19, wherein analyzing the data comprises evaluating the data for one or more biomarkers indicative of the neurodegenerative disease.

Clause 21. The method of any one of clauses 1-20, wherein the one or more biomarkers comprise Amyloid or Tau protein formations.

Clause 22. The method of any one of clauses 1-21, wherein the neurodegenerative disease is selected from the group consisting of Alzheimer's disease, Parkinson's disease, Amyotrophic Lateral Sclerosis, Multiple Sclerosis, Prion disease, Motor neurone diseases (MND), Huntington's disease (HD), Spinocerebellar ataxia (SCA), Spinal muscular atrophy (SMA), cerebral amyloid angiopathy (CAA).

Clause 23. A system, comprising: a light source configured to illuminate an eye of a patient with light: an imaging device configured to receive light returned from the eye to generate a spectropolarimetric image of the eye, the spectropolarimetric image comprising a plurality of pixels and comprising, for each pixel, spatial, spectral, and polarimetric data; and a computing device configured to: receive the spectropolarimetric image: analyze the spatial, spectral, and polarimetric data for the plurality of pixels: based on the analyzing, classify the patient into at least one category of a plurality of categories, each category of the plurality of categories indicating a status with respect to a neurodegenerative disease that affects a central nervous system; and providing one or more of the at least one category as an output to indicate the status of the patient with respect to the neurodegenerative disease.

Clause 24. The system of clause 23, further comprising generating the spatial, spectral, and polarimetric data synchronously as a single data package of spectropolarimetric data.

Clause 25. The system of clause 23 or clause 24, wherein: the spectropolarimetric image comprises a multi-dimensional spectropolarimetric data package: analyzing the spatial, spectral, and polarimetric data comprises applying one or more neural networks to each dimension of the multi-dimensional spectropolarimetric data package to generate a dimensional output for each dimension of the multi-dimensional spectropolarimetric data package; and classifying the patient into the at least one category comprises combining each dimensional output of each dimension of the multi-dimensional spectropolarimetric data package.

Clause 26. The system of any one of clauses 23-25, wherein analyzing the spatial, spectral, and polarimetric data comprises: receiving a segmentation measurement of one or more regions of the eye; and receiving, from the imaging device, a multi-dimensional spectropolarimetric measurement of the eye.

Clause 27. The system of any one of clauses 23-26, wherein classifying the patient into the at least one category comprises applying one or more classification networks to the segmentation measurement and the multi-dimensional spectropolarimetric measurement to select a disease classification of the eye.

Clause 28. The system of any one of clauses 23-27, wherein spatial, spectral, and polarimetric data comprises spectropolarimetric data packages comprising spectropolarimetric components relating to an anatomical location of the eye.

Clause 29. The system of any one of clauses 23-28, wherein the status of the patient with respect to the neurodegenerative disease comprises a risk of the patient having or experiencing symptoms related to the neurodegenerative disease.

Clause 30. The system of any one of clauses 23-29, wherein the status of the patient with respect to the neurodegenerative disease comprises a diagnosis of the patient as having the neurodegenerative disease.

Clause 31. The system of any one of clauses 23-30, wherein the status of the patient with respect to the neurodegenerative disease comprises a progression of the neurodegenerative disease in the patient.

Clause 32. The system of any one of clauses 23-31, wherein the status of the patient with respect to the neurodegenerative disease comprises a response of the patient to preventative interventions or treatment interventions.

Clause 33. The system of any one of clauses 23-32, wherein analyzing the spatial, spectral, and polarimetric data comprises analyzing the spatial, spectral, and polarimetric data for the plurality of pixels with a regression model.

Clause 34. The system of any one of clauses 23-33, wherein classifying the patient into the at least one category comprises classifying the patient based on a plurality of pathologies of the neurodegenerative disease.

Clause 35. The system of any one of clauses 23-34, wherein classifying the patient based on the plurality of pathologies comprises classifying the patient based on a combined weighted score, scorecard, or probabilistic determination of each of the plurality of pathologies.

Clause 36. The system of any one of clauses 23-35, wherein analyzing the spatial, spectral, and polarimetric data comprises: performing semantic segmentation to identify different parts of the eye; and combining the semantic segmentation with the spatial, spectral, and polarimetric data for the plurality of pixels.

Clause 37. The system of any one of clauses 23-36, wherein analyzing the spatial, spectral, and polarimetric data further comprises analyzing a relationship between the spatial, spectral, and polarimetric data for a first pixel with the spatial, spectral, and polarimetric data of two more adjacent pixels.

Clause 38. The system of any one of clauses 23-37, wherein analyzing the spatial, spectral, and polarimetric data comprises analyzing the spatial, spectral, and polarimetric data for the plurality of pixels with an ensemble prediction model.

Clause 39. The system of any one of clauses 23-38, wherein each individual model of the ensemble prediction model is assigned a weight based on how significantly a prediction from each individual model correlates with amyloid or tau status of the patient.

Clause 40. The system of any one of clauses 23-39, wherein analyzing the spatial, spectral, and polarimetric data comprises analyzing the spatial, spectral, and polarimetric data for the plurality of pixels with a convolutional neural network to generate a heatmap.

Clause 41. The system of any one of clauses 23-40, wherein analyzing the spatial, spectral, and polarimetric data comprises calculating a quality assurance criterion for each of the plurality of pixels.

Clause 42. The system of any one of clauses 23-41, wherein analyzing the spatial, spectral, and polarimetric data comprises evaluating the data for one or more biomarkers indicative of the neurodegenerative disease.

Clause 43. The system of any one of clauses 23-42, wherein the one or more biomarkers comprise Amyloid or Tau protein formations.

Clause 44. The system of any one of clauses 23-43, wherein the neurodegenerative disease is selected from the group consisting of Alzheimer's disease, Parkinson's disease, Amyotrophic Lateral Sclerosis, Multiple Sclerosis, Prion disease, Motor neurone diseases (MND), Huntington's disease (HD), Spinocerebellar ataxia (SCA), Spinal muscular atrophy (SMA), cerebral amyloid angiopathy (CAA).

The preceding description provides exemplary embodiments only, and is not intended to limit the scope, applicability, or configuration of the disclosure. Rather, the preceding description of the exemplary embodiments will provide those skilled in the art with an enabling description for implementing one or more exemplary embodiments. It will be understood that various changes may be made in the function and arrangement of elements without departing from the spirit and scope of the presently disclosed embodiments.

From the foregoing description, it will be apparent that variations and modifications may be made to the embodiments of the present disclosure to adopt it to various usages and conditions. Such embodiments are also within the scope of the following claims.

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method comprising:
analyzing data from an imaging of an eye of a patient with at least one processor, the data from the imaging comprising a plurality of pixels and comprising, for each pixel, spatial, spectral, and polarimetric data generated from the imaging of the eye of the patient and wherein analyzing the data comprises analyzing the spatial, spectral, and polarimetric data for the plurality of pixels;
based on the analyzing, with the at least one processor, classifying the patient into at least one category of a plurality of categories, each category of the plurality of categories indicating a status with respect to a neurodegenerative disease that affects a central nervous system; and
generating, with the at least one processor, an output of one or more of the at least one category indicating the status of the patient with respect to the neurodegenerative disease that affects a central nervous system.

2. The method of claim 1, further comprising generating the spatial, spectral, and polarimetric data synchronously as a single data package of spectropolarimetric data.

3. The method of claim 1, wherein:
the data from the imaging comprises a multi-dimensional spectropolarimetric data package; and
classifying the patient into the at least one category comprises:
applying, by the at least one processor, one or more neural networks to each dimension of the multi-dimensional spectropolarimetric data package to generate a dimensional output for each dimension of the multi-dimensional spectropolarimetric data package; and
generating, by the at least one processor, the output as a disease classification of the multi-dimensional spectropolarimetric data package by combining each dimensional output of each dimension of the multi-dimensional spectropolarimetric data package.

4. The method of claim 1, wherein analyzing the data from the imaging of the eye comprises:
receiving, by the at least one processor, a segmentation measurement of one or more regions of the eye; and
receiving, by the at least one processor, from a spectropolarimetric camera, a multi-dimensional spectropolarimetric measurement of the eye.

5. The method of claim 4, wherein classifying the patient into the at least one category comprises:
applying, by the at least one processor, one or more classification networks to the segmentation measurement and the multi-dimensional spectropolarimetric measurement to select a disease classification of the eye.

6. The method of claim 1, wherein the data from the imaging of the eye comprises spectropolarimetric data packages comprising spectropolarimetric components relating to an anatomical location of the eye.

7. The method of claim 1, wherein the status of the patient with respect to the neurodegenerative disease comprises a risk of the patient having or experiencing symptoms related to the neurodegenerative disease.

8. The method of claim 1, wherein the status of the patient with respect to the neurodegenerative disease comprises a diagnosis of the patient as having the neurodegenerative disease.

9. The method of claim 1, wherein the status of the patient with respect to the neurodegenerative disease comprises a progression of the neurodegenerative disease in the patient.

10. The method of claim 1, wherein the status of the patient with respect to the neurodegenerative disease comprises a response of the patient to preventative interventions or treatment interventions.

11. The method of claim 1, wherein analyzing the data comprises analyzing the spatial, spectral, and polarimetric data for the plurality of pixels with a regression model.

12. The method of claim 1, wherein classifying the patient into the at least one category comprises classifying the patient based on a plurality of pathologies of the neurodegenerative disease.

13. The method of claim 12, wherein classifying the patient based on the plurality of pathologies comprises classifying the patient based on a combined weighted score, scorecard, or probabilistic determination of each of the plurality of pathologies.

14. The method of claim 1, wherein analyzing the data from the imaging of the eye comprises:
performing semantic segmentation to identify different parts of the eye; and
combining the semantic segmentation with the spatial, spectral, and polarimetric data for the plurality of pixels.

15. The method of claim 1, wherein analyzing the data further comprises analyzing a relationship between the spatial, spectral, and polarimetric data for a first pixel with the spatial, spectral, and polarimetric data of two more adjacent pixels.

16. The method of claim 1, wherein analyzing the data comprises analyzing the spatial, spectral, and polarimetric data for the plurality of pixels with an ensemble prediction model.

17. The method of claim 16, wherein each individual model of the ensemble prediction model is assigned a weight based on how significantly a prediction from each individual model correlates with amyloid or tau status of the patient.

18. The method of claim 1, wherein analyzing the data comprises analyzing the spatial, spectral, and polarimetric data for the plurality of pixels with a convolutional neural network to generate a heatmap.

19. The method of claim 1, wherein analyzing the data comprises calculating a quality assurance criterion for each of the plurality of pixels.

20. The method of claim 1, wherein analyzing the data comprises evaluating the data for one or more biomarkers indicative of the neurodegenerative disease.

21. The method of claim 20, wherein the one or more biomarkers comprise Amyloid or Tau protein formations.

22. The method of claim 1, wherein the neurodegenerative disease is selected from the group consisting of Alzheimer's disease, Parkinson's disease, Amyotrophic Lateral Sclerosis, Multiple Sclerosis, Prion disease, Motor neurone diseases (MND), Huntington's disease (HD), Spinocerebellar ataxia (SCA), Spinal muscular atrophy (SMA), cerebral amyloid angiopathy (CAA).

23. A system, comprising:
a light source configured to illuminate an eye of a patient with light;
an imaging device configured to receive light returned from the eye to generate a spectropolarimetric image of the eye, the spectropolarimetric image comprising a plurality of pixels and comprising, for each pixel, spatial, spectral, and polarimetric data; and
a computing device configured to:
receive the spectropolarimetric image;
analyze the spatial, spectral, and polarimetric data for the plurality of pixels;
based on the analyzing, classify the patient into at least one category of a plurality of categories, each category of the plurality of categories indicating a status with respect to a neurodegenerative disease that affects a central nervous system; and
providing one or more of the at least one category as an output to indicate the status of the patient with respect to the neurodegenerative disease.

24.-44. (canceled)
